(19) United States
(12) Patent Application Publication
(10) Pub. No.: US 2023/0312742 A1
(43) Pub. Date: Oct. 5, 2023

(54) CD38 antibodies for the treatment of human diseases

(71) Applicant: Y-mAbs Therapeutics, Inc., New York, NY (US)

(72) Inventors: Ahmed MAHIUDDIN, Verona, NJ (US); Linlin WANG, Mountain Lakes, NJ (US); Sayed Shahabuddin HOSEINI, New York, NY (US); Ole BAADSGAARD, Hellerup (DK); Claus J. MØLLER SAN-PEDRO, Gilleleje (DK)

(21) Appl. No.: 18/008,581

(22) PCT Filed: Jun. 14, 2021
(86) PCT No.: PCT/DK2021/050188

§ 371 Date: Dec. 6, 2022

Related U.S. Application Data
(60) Provisional application No. 63/040,220, filed on Jun. 17, 2020.

(51) Int. Cl. C07K 16/28; C07K 16/44; A61K 51/10

(52) U.S. Cl. CPC C07K 16/2896; C07K 16/44; A61K 51/1027; C07K 2319/00; C07K 2317/622; C07K 2317/24; C07K 2317/92

(57) ABSTRACT

The present invention relates to antibodies and antigen binding fragment thereof, capable of binding to CD38 antigen. More specifically, the invention relates to antibodies and antigen binding fragments, wherein said antibodies or antigen binding fragments comprises sequences of human origin, and wherein said sequences of human origin reduces immunogenicity compared to a murine antibody. The invention further relates to bispecific antibodies for binding to CD38 and CD3. Further, the invention relates to bispecific antibodies for binding to CD38 and a chelator. The invention further relates to antibodies and antigen binding fragments for the treatment of cancer and autoimmune diseases.

The present invention relates to antibodies and antigen binding fragments thereof, capable of binding to CD38 antigen. More specifically, the invention relates to antibodies and antigen binding fragments, wherein said antibodies or antigen binding fragments comprises sequences of human origin, and wherein said sequences of human origin reduces human immunogenicity compared to a murine antibody. The invention further relates to bispecific antibodies for binding to CD38 and CD3. Further, the invention relates to bispecific antibodies for binding to CD38 and a chelator. The invention further relates to antibodies and antigen binding fragments for the treatment of cancer and autoimmune diseases.

### TECHNICAL BACKGROUND

According to Lee 2006 and van de Donk 2018, CD38 is a transmembrane glycoprotein with enzymatic, adhesion and receptor functions expressed at low levels on hematopoietic and some non-hematopoietic tissues [1, 2]. Morandi 2018 describes Multiple myeloma (MM), the neoplastic proliferation of plasma cells, as the second most common hematologic cancer that accounts for 1% of all human cancers [3]. According to Lin 2004 CD38 is expressed on almost 100% of MM cells at high levels [4]. Chiaretti 2009 describes T-cell acute lymphoblastic leukemia (T-ALL) to account for 15 to 25% of ALL cases in children and adults [5]. According to Raetz 2016 T-ALL patients relapse in 20% of cases with a poor survival of 25% [6]. CD38 is also described by Naik 2019 to be overexpressed on the majority of T-ALL cases [7].

According to van de Donk 2018, Feng 2017, Esteve-Sole 2017 and Chen 2018, CD38 is, in addition to its expression on various tumor cells, also expressed on regulatory T and B cells (Treg and Breg) as well as on myeloid derived suppressor cells (MDSC) and exhausted T cells [2, 8-10].

The following antibody agents are described to target CD38:
Daratumumab: This antibody is presently the only FDA-approved antibody against CD38 (Daratumumab, DARZALEX; Janssen Biotech). According to the FDA label [1] Daratumumab is human IgG1k monoclonal antibody against CD38 for treatment of multiple myeloma. It is used for treatment of MM and also as an immunomodulatory agent. Daratumumab has been used as a monotherapy for patients with MM as described in the FDA label. According to the FDA label [1] the overall response rate (ORR) was 31%; however, they were partial responses and no complete response (CR) was observed [16]. In another similar study described by the FDA label [1], the ORR was 36% with only 2.3% CR [16].
MOR202 (MOR03087): According to Raab 2016 MOR202 is a human IgG1 CD38 monoclonal antibody in clinical trials for MM treatment. In a phase II clinical trial, it is described to induce 40% responses (partial responses or stable disease) but no CR was reported [13].
AMG 424: According to Zuch de Zafra AMG 424 is a Fab/scFv-Fc T-cell engaging bispecific antibody against CD38. No clinical data has been reported regarding this antibody that is in a phase I clinical trial [15].
GBR 1342: According to Glen mark pharma GBR 1342 is a Fab/scFv-Fc T-cell engaging bispecific antibody against CD38 [18]. No clinical data has been reported regarding this antibody, that is in a phase I clinical trial.

### SUMMARY OF THE INVENTION

Overexpression of CD38 on several human tumors indicates it is a suitable target for immunotherapy.

T-cell methodology have emerged as one of the most successful approaches for leukemia treatment. Redirection of T cells using chimeric antigen receptors (CAR) and bispecific antibodies (BsAb) against CD19(+) malignancies has led to promising clinical trial results and FDA approval of Yescarta™, Kymriah™, and Blincyto™[19, 20, 21]. Development of T-cell engaging bispecific antibodies against other tumor-associated antigens such as CD38 is very much needed to mitigate poor outcomes of patients with MM and other CD38-related malignancies.

Targeting cancer with antibodies conjugated to radioisotopes is an active arena of cancer drug discovery. Several radioimmunotherapy agents have been approved or are in clinical trials mainly for treatment of hematological malignancies and also solid cancers [11]. Since CD38 gets internalized upon binding to anti-CD38 antibodies, it is a good target for radioimmunotherapy [12].

Targeting CD38 is a relevant strategy to treat CD38(+) human tumors including MM and T-ALL. Targeting CD38 may also be a relevant approach to eliminate immune suppressing tumor microenvironment in various solid or hematologic cancers.

Few anti-CD38 antibodies are in clinical development but there is only one antibody that is FDA-approved. Therefore, there is an unmet need to develop novel anti-CD38 agents for human diseases.

Here, we report generation of an IgG-L-scFv BsAb with bivalent binding to CD38 and CD3. In addition, a CD38-specific radioimmunotherapy agent is also presented here.

AT13/5 is a murine anti-CD38 antibody, which was generated in 1995 [14]. It can be supplied by ThermoFisher scientific [17]

U.S. Pat. No. 7,829,673B2 describes isolated human monoclonal antibodies which bind to human CD38 and related antibody-based compositions and molecules. According to U.S. Pat. No. 7,829,673B2, the invention relates to antibodies which have specific characteristics and which are useful for treating inter alia multiple myeloma.

According to an embodiment, the invention concerns an antibody or antigen binding fragment that comprises a SADA construct. In certain embodiments, the SADA antibody comprises a single-chain variable fragment (scFv) against CD38 and a second scFv against DOTA (based on humanized C825 antibody from Patent WO2016130539A2 all of which is incorporated by reference in its entirety). The SADA constructs assemble in tetramers and bind to the tumor target in vivo. Unbound constructs predictably disassemble into smaller antibody fragments and are excreted through the kidneys within hours after administration without using clearing agents. This technology provided promising treatment with higher uptake of payload at tumor and lower toxicity.

In certain embodiments, the antibody or antigen binding fragment thereof is linked to a self-assembly disassembly (SADA) polypeptide disclosed in International Patent Application Publication No. WO2018204873, all of which is incorporated by reference in its entirety.

In certain embodiments, the antibody or antigen binding fragment thereof comprises an engineered protein with high affinity for DOTA chelates, disclosed in US patent no. U58648176 or International Patent Application Publication No. WO2010099536 all of which is incorporated by reference in its entirety.

According to an aspect, the invention concerns, an antibody or antigen binding fragment thereof, capable of binding to CD38 antigen, wherein said antibody or antigen binding fragment comprises at least one sequence selected among a heavy chain variable region CDR1 according to SEQ ID No. 34, a heavy chain variable region CDR2 according to SEQ IN No. 35, a heavy chain variable region CDR3 according to SEQ IN No. 36, a light chain variable region CDR1 according to SEQ ID No. 31, a light chain variable region CDR2 according to SEQ ID No. 32 and a light chain variable region CDR3 according to SEQ ID No. 33, wherein said antibody comprises sequences of human origin.

According to another aspect, the invention concerns an antibody or antigen binding fragment thereof, preferably according to the invention, capable of binding to CD38 antigen, wherein said antibody or antigen binding fragment comprises at least one sequence having at least 70%, at least 75%, at least 80% or preferably at least 85% identity to at least one sequence selected among any of the sequences SEQ ID No. 18, 19, 60 and 61. According to another aspect, the invention concerns an antibody or antigen binding fragment thereof, capable of binding to CD38 antigen, wherein said antibody or antigen binding fragment comprises at least one sequence selected among sequence ID No. 31-36 and wherein said antibody or antigen binding fragment comprises at least one sequence having at least 70%, preferably at least 85% identity to any of the sequences SEQ ID No. 18, 19, 60 and 61.

According to another aspect, the invention concerns an antibody or antigen binding fragment thereof, capable of binding to CD38 antigen, wherein said antibody or antigen binding fragment comprises a heavy chain or variable heavy chain sequence that has at least about 80%, about 81%, about 82%, about 83%, about 84%, about 85%, about 86%, about 87%, about 88%, about 89%, about 90%, about 91% about 92%, about 93%, about 94%, about 95%, about 96%, about 97%, about 98% or about 99% identity to a sequence selected among the sequences set forth in SEQ ID No. 8-17, 21, 23, 25, 39, 40 and 53 and/or a light chain or variable light chain sequence that has at least about 80%, about 81%, about 82%, about 83%, about 84%, about 85%, about 86%, about 87%, about 88%, about 89%, about 90%, about 91% about 92%, about 93%, about 94%, about 95%, about 96%, about 97%, about 98% or about 99% sequence identity to a sequence selected among the sequences set forth in SEQ ID No. 1-7, 20, 22, 24, 37, 38 and 54.

According to an aspect, the invention concerns an antibody or antigen binding fragment thereof, preferably according to the invention, capable of binding to CD38 antigen, wherein said antibody or antigen binding fragment comprises a heavy chain or variable heavy chain sequence selected among the sequences set forth in SEQ ID No. 8-17, 21, 23, 25, 39, 40 and 53 and/or a light chain or variable light chain sequence selected among the sequences set forth in SEQ ID No. 1-7, 20, 22, 24, 37, 38 and 54.

According to another aspect, the invention concerns an antibody or antigen binding fragment thereof, capable of binding to CD38 antigen, wherein said antibody or antigen binding fragment comprises a sequence that has at least about 80%, about 81%, about 82%, about 83%, about 84%, about 85%, about 86%, about 87%, about 88%, about 89%, about 90%, about 91% about 92%, about 93%, about 94%, about 95%, about 96%, about 97%, about 98% or about 99% identity to a sequence selected among the sequences set forth in SEQ ID No. 20-30.

According to an aspect, the invention concerns an antibody or antigen binding fragment thereof, preferably according to the invention, capable of binding to CD38 antigen, wherein said antibody or antigen binding fragment comprises a sequence selected from the group consisting of SEQ ID No. 26-30.

According to another aspect, the invention concerns a self-assembly disassembly (SADA) polypeptide, wherein said polypeptide is linked to an antibody or antigen binding fragment according to the invention.

According to another aspect, the invention concerns a polypeptide conjugate comprising a self-assembly disassembly (SADA) polypeptide according to the invention, and an antibody or antigen binding fragment according to the invention.

According to another aspect, the invention concerns a polypeptide conjugate comprising a self-assembly disassembly (SADA) polypeptide, and wherein said conjugate further comprises the bispecific antibody according to the invention, wherein said first antigen is CD38 and wherein said second antigen is DOTA.

DOTA (Dodecane Tetraacetic Acid) is also referred to as 1,4,7,10-tetraazacyclododecane-1,4,7 10-tetraacetic acid, and has the formula (CH₂CH₂NCH₂CO₂H)₄.

DTPA (Diethylene Triamine Pentaacetic Acid) is also referred to with the IUPAC name 2-[bis[2-[bis(carboxymethyl)amino]ethyl]amino]acetic acid. DTPA has the molecular formula C₁₄H₂₃N₃O₁₀.

According to another aspect, the invention concerns a polypeptide conjugate comprising a self-assembly disassembly (SADA) polypeptide, and at least a first binding domain that binds to a first target and is covalently linked to the SADA polypeptide.

According to another aspect, the invention concerns an isolated nucleic acid molecule encoding the antibody or antigen binding fragment according to the invention.

According to another aspect, the invention concerns an isolated nucleic acid molecule encoding an antibody or antigen binding fragment according to the invention.

According to another aspect, the invention concerns a recombinant vector comprising the isolated nucleic acid molecule according to the invention.

According to another aspect, the invention concerns a host cell comprising the recombinant vector according to the invention.

According to another aspect, the invention concerns a method for the production of an antibody or antigen binding fragment thereof according to the invention comprising a step of culturing the host cell according to the invention in a culture medium under conditions allowing the expression of the antibody or fragment and separating the antibody or fragment from the culture medium.

According to another aspect, the invention concerns a chimeric antigen receptor (CAR) comprising an antibody or antigen binding fragment according to the invention.

According to another aspect, the invention concerns a CAR-T cell expressing a CAR according to the invention.

According to another aspect, the invention concerns a population of CAR-T cells.

According to another aspect, the invention concerns a composition comprising the population of CAR-T cells according to the invention.

According to another aspect, the invention concerns a CAR-NK cell expressing a CAR according to the invention.

According to another aspect, the invention concerns a population of CAR-NK cells.

According to another aspect, the invention concerns a composition comprising the population of CAR-NK cells according to the invention.

According to another aspect, the invention concerns a pharmaceutical composition comprising the antibody or antigen binding fragment according to the invention.

According to another aspect, the invention concerns a T cell armed with the antibody or antigen binding fragment according to the invention.

According to another aspect, the invention concerns a method of treating, preventing, alleviating and/or diagnosing the symptoms of a medical condition in a subject, comprising a step of administration of an antibody, an antigen binding fragment, a bispecific antibody, a trispecific antibody, a polypeptide conjugate, a composition and/or a CAR to a subject, and wherein said medical condition is characterized by expression of CD38 antigen.

According to another aspect, the invention concerns use of the composition according to the invention in the manufacturing of a medicament for the treatment of a cancer, for use in a method according to the invention.

According to another aspect, the invention concerns use of the antibody or antigen binding fragment according to the invention in the manufacturing of a medicament for the treatment of a cancer and/or for use in a method according to the invention.

According to another aspect, the invention concerns an in vitro use of an antibody or antigen binding fragment thereof according to the invention.

### DETAILED DISCLOSURE

According to an embodiment, the invention concerns a n antibody or antigen binding fragment thereof, capable of binding to CD38 antigen, wherein said antibody or antigen binding fragment comprises at least one sequence selected among a heavy chain variable region CDR1 according to SEQ ID No. 34, a heavy chain variable region CDR2 according to SEQ IN No. 35, a heavy chain variable region CDR3 according to SEQ IN No. 36, a light chain variable region CDR1 according to SEQ ID No. 31, a light chain variable region CDR2 according to SEQ ID No. 32 and a light chain variable region CDR3 according to SEQ ID No. 33, wherein said antibody comprises sequences of human origin.

Immunogenicity may be defined as the ability of a substance to provoke an immune response in the body of a subject.

According to an embodiment, the invention concerns a n antibody or antigen binding fragment thereof, preferably according to the invention, capable of binding to CD38 antigen, wherein said antibody or antigen binding fragment comprises at least one sequence having at least 70%, at least 75%, at least 80% or preferably at least 85% identity to at least one sequence selected among any of the sequences SEQ ID No. 18, 19, 60 and 61.

According to an embodiment, the invention concerns a n antibody or antigen binding fragment thereof, capable of binding to CD38 antigen, wherein said antibody or antigen binding fragment comprises at least one sequence selected among sequence ID No. 31-36 and wherein said antibody or antigen binding fragment comprises at least one sequence having at least 70%, preferably at least 85% identity to any of the sequences SEQ ID No. 18, 19,60 and 61.

According to an embodiment, the invention concerns a n antibody or antigen binding fragment thereof, capable of binding to CD38 antigen, wherein said antibody or antigen binding fragment comprises a heavy chain sequence or variable heavy chain that has at least about 80%, about 81%, about 82%, about 83%, about 84%, about 85%, about 86%, about 87%, about 88%, about 89%, about 90%, about 91% about 92%, about 93%, about 94%, about 95%, about 96%, about 97%, about 98% or about 99% identity to a sequence selected among the sequences set forth in SEQ ID No. 8-17, 21, 23, 25, 39, 40 and 53 and/or a light chain or variable light chain sequence that has at least about 80%, about 81%, about 82%, about 83%, about 84%, about 85%, about 86%, about 87%, about 88%, about 89%, about 90%, about 91% about 92%, about 93%, about 94%, about 95%, about 96%, about 97%, about 98% or about 99% sequence identity to a sequence selected among the sequences set forth in SEQ ID No. 1-7, 20, 22, 24, 37, 38 and 54.

According to an embodiment, the invention concerns an antibody or antigen binding fragment thereof, preferably according to the invention, capable of binding to CD38 antigen, wherein said antibody or antigen binding fragment comprises a heavy chain or variable heavy chain sequence selected among the sequences set forth in SEQ ID No. 8-17, 21, 23, 25, 39, 40 and 53 and/or a light chain or variable light chain sequence selected among the sequences set forth in SEQ ID No. 1-7, 20, 22, 24, 37, 38 and 54.

According to an embodiment, the invention concerns an antibody or antigen binding fragment thereof, capable of binding to CD38 antigen, wherein said antibody or antigen binding fragment comprises a sequence that has at least about 80%, about 81%, about 82%, about 83%, about 84%, about 85%, about 86%, about 87%, about 88%, about 89%, about 90%, about 91% about 92%, about 93%, about 94%, about 95%, about 96%, about 97%, about 98% or about 99% identity to a sequence selected among the sequences set forth in SEQ ID No. 20-30.

According to an embodiment, the invention concerns an antibody or antigen binding fragment thereof, preferably according to the invention, capable of binding to CD38 antigen, wherein said antibody or antigen binding fragment comprises a sequence selected from the group consisting of SEQ ID No. 26-30.

According to an embodiment, the invention concerns the antibody or antigen binding fragment, wherein said sequences of human origin reduces immunogenicity as compared to a murine antibody.

Immunogenicity may be defined as the ability of a substance to provoke an immune response in the body of a subject.

In certain embodiments said murine antibody comprises the murine variable regions (VL and VH) of AT13/5 anti-CD38 antibody.

In certain embodiments said murine antibody comprises a light chain sequence according to SEQ ID No. 1, and a heavy chain sequence according to sequence ID No. 8.

According to an embodiment, the invention concerns the antibody or antigen binding fragment, wherein said antibody comprises a heavy chain sequence comprising a sequence according to SEQ ID No. 8-17, 21, 23, 25, 39, 40 and 53 and/or a light chain sequence comprising a sequence according to SEQ ID No. 1-7, 20, 22, 24, 37, 38 and 54.

According to an embodiment, the invention concerns the humanized antibody or antigen binding fragment thereof, comprising Fc, Fc2 or Null-Fc.

According to an embodiment, the invention concerns the antibody or antigen binding fragment, wherein said antibody comprises a heavy chain sequence that is at least about 80%, about 81%, about 82%, about 83%, about 84%, about 85%, about 86%, about 87%, about 88%, about 89%, about 90%, about 91% about 92%, about 93%, about 94%, about 95%, about 96%, about 97%, about 98% or about 99% sequence identity to the sequence set forth in SEQ ID No. 8-17, 21, 23, 25, 39, 40 and 53 and/or a light chain sequence that is at least about 80%, about 81%, about 82%, about 83%, about 84%, about 85%, about 86%, about 87%, about 88%, about 89%, about 90%, about 91% about 92%, about 93%, about 94%, about 95%, about 96%, about 97%, about 98% or about 99% sequence identity to the sequence set forth in SEQ ID No. 1-7, 20, 22, 24, 37, 38 and 54.

According to an embodiment, the invention concerns the antibody or antigen binding fragment, wherein said antibody or antigen binding fragment comprises at least one sequence having at least 50%, at least 55%, at least 60%, at least 65%, at least 70%, at least 75%, at least 77.5%, at least 80%, at least 82%, at least 84%, at least 86%, at least 88% or at least 90%, at least 92% or at least 94% identity to at least one sequence selected among any of the sequences 18, 19, 60 and 61.

According to an embodiment, the invention concerns the antibody or antigen binding fragment, wherein said antibody or antigen binding fragment binds to an epitope, and wherein said epitope is an epitope of CD38.

According to an embodiment, the invention concerns the antibody or antigen binding fragment, wherein said antibody of antigen binding fragment binds to a sequence selected among the sequences according to SEQ ID No.: 56 and 57.

According to an embodiment, the invention concerns the antibody or antigen binding fragment, wherein said antigen is present on a cancer cell.

According to an embodiment, the invention concerns the antibody or antigen binding fragment, wherein said cancer cells is from a metastasis.

According to an embodiment, the invention concerns the antibody or antigen binding fragment, wherein said cancer cells is from an adult cancer.

According to an embodiment, the invention concerns the antibody or antigen binding fragment, wherein said cancer cells is from a pediatric tumor.

According to an embodiment, the invention concerns the antibody or antigen binding fragment, wherein said cancer cells and/or metastasis is a solid tumor.

According to an embodiment, the invention concerns the antibody or antigen binding fragment, wherein said cancer cells and/or metastasis is selected among a multiple myeloma (MM), an AL amyloidosis, an Acute myeloid leukemia (AML), a myelodysplastic syndrome (MDS), a proliferative glomerulonephritis with monoclonal immune deposit, a C3 glomerulopathy associated with monoclonal gammopathy, a squamous cell carcinoma, a colon cancer, a non-small cell lung cancer, a mantle cell lymphoma, a diffuse large B-cell lymphoma, a follicular lymphoma, a NK-/T-cell lymphoma, a B-cell non-Hodgkin lymphoma, a T-cell acute lymphoblastic leukemia, a B-cell acute lymphoblastic leukemia, a chronic lymphocytic leukemia (CLL), a Waldenström macroglobulinemia, a solid cancer, a cancer with immunomodulatory activity and a cancer expressing CD38.

According to an embodiment, the invention concerns the antibody or antigen binding fragment, wherein said antibody or antigen binding fragment is for use in the treatment of an autoimmune disease.

According to an embodiment, the invention concerns the antibody or antigen binding fragment, wherein said autoimmune disease is selected among a rheumatoid arthritis, multiple sclerosis, paraneoplastic syndromes, systemic lupus erythematosus, type 2 diabetes, Pemphigus Vulgaris, autoimmune hemolytic anemia, allergy, and graft-versus-host disease.

According to an embodiment, the invention concerns the antibody or antigen binding fragment, wherein said antibody or antigen binding fragment comprises an Fc.

According to an embodiment, the invention concerns the antibody or antigen binding fragment, comprising a Fc region which does not interact with a Fc gamma receptor.

According to an embodiment, the invention concerns the antibody or antigen binding fragment, further comprising an Fc region, wherein said Fc region is not reactive or exhibit little reactivity.

According to an embodiment, the invention concerns the antibody or antigen binding fragment, wherein said antibody comprises a null Fc.

According to an embodiment, the invention concerns the antibody or antigen binding fragment, wherein said Fc comprises any of the mutations N297A, K322A, L234A and/or L235A.

According to an embodiment, the invention concerns the antibody or antigen binding fragment thereof, wherein said antibody or antigen binding fragment has an immunogenicity of less than 50%, less than 45%, less than 40%, less than 35%, less than 30%, less than 25%, less than 20%, less than 15% or about 10% as compared to a murine antibody.

According to an embodiment, the invention concerns the antibody or antigen binding fragment thereof, wherein said antibody or antigen binding fragment is a murine antibody or an antigen binding fragment thereof.

According to an embodiment, the invention concerns the antibody or antigen binding fragment thereof, wherein said antibody or antigen binding fragment is a chimeric antibody or an antigen binding fragment thereof.

According to an embodiment, the invention concerns the antibody or antigen binding fragment thereof, wherein said antibody or antigen binding fragment is a humanized antibody or an antigen binding fragment thereof.

According to an embodiment, the invention concerns the antibody or antigen binding fragment thereof, wherein said antibody or antigen binding fragment is radiolabeled with a radioactive isotope.

According to an embodiment, the invention concerns the antibody or antigen binding fragment thereof, wherein said radioactive isotope is selected among ²¹¹At, ¹⁴C, ⁵¹Cr, ⁵⁷Co, ⁵⁸Co, ⁶⁷Cu, ¹⁵²Eu, ⁶⁷Ga, ³H, ¹¹¹In, ⁵⁹Fe, ²¹²Pb, ¹⁷⁷Lu, ³²P, ²²³Ra, ²²⁴Ra, ¹⁸⁶Re, ¹⁸⁷Re, ¹⁸⁸Re, ⁷⁵Se, ³⁵S, ^{99m}Tc, ²²⁷Th, ⁸⁹Zr, ⁹⁹Y, ¹²³I, ¹²⁴I, ¹²⁵I, ¹³¹I, ¹³⁵I, ^{94m}Tc, ⁶⁴Cu, ⁶⁸Ga, ⁶⁶Ga, ⁷⁶Br, ⁸⁶Y, ⁸²Rb, ^{110m}In, ¹³N, ¹¹C, ⁹⁰Y, ^{99m}Tc, ⁸⁹Zr, ¹⁹F and ¹⁸F.

According to an embodiment, the invention concerns the antibody or antigen binding fragment thereof, wherein said radioactive isotope is selected among a PET label and or a SPECT label.

According to an embodiment, the invention concerns the antibody or antigen binding fragment thereof, wherein said PET label is selected among ¹²⁴I, ²²⁵Ac and ⁸⁹Zr.

According to an embodiment, the invention concerns the antibody or antigen binding fragment thereof, wherein said SPECT label is selected among ¹³¹I, ¹⁷⁷Lu, ^{99m}Tc, ⁶⁴Cu and ⁸⁹Zr.

According to an embodiment, the invention concerns the antibody or antigen binding fragment thereof, wherein said antibody or antigen binding fragment is conjugated to a chelator compound.

According to an embodiment, the invention concerns the antibody or antigen binding fragment thereof, wherein said chelator compound is bound to a radioactive isotope.

According to an embodiment, the invention concerns the antibody or antigen binding fragment thereof, wherein said radioactive isotope is selected among ³H, ¹⁴C, ¹⁸F, ¹⁹F, ³²P, ³⁵S, ¹³⁵I, ¹²⁵I, ¹²⁴I, ¹²³I, ¹³¹I, ⁶⁴Cu, ¹⁸⁷Re, ¹¹¹In, ⁹⁰Y, ^{99m}Tc, ¹⁷⁷Lu and ⁸⁹Zr.

According to an embodiment, the invention concerns the antibody or antigen binding fragment thereof, wherein said chelator compound is selected among DOTA, DTPA, NOTA and DFO.

According to an embodiment, the invention concerns the antibody or antigen binding fragment thereof, wherein said DOTA is a variant of DOTA, such as Benzyl-DOTA.

DOTA (Dodecane Tetraacetic Acid) is also referred to as 1,4,7,10-tetraazacyclododecane-1,4,7 10-tetraacetic acid, and has the formula (CH₂CH₂NCH₂CO₂H)₄.

DTPA (Diethylene Triamine Pentaacetic Acid) is also referred to with the IUPAC name 2-[bis[2-[bis(carboxymethyl)amino]ethyl]amino]acetic acid. DTPA has the molecular formula C₁₄H₂₃N₃O₁₀.

According to an embodiment, the invention concerns the antibody or antigen binding fragment thereof, wherein said DTPA is a variant of DTPA, such as CHX-A″-DTPA.

According to an embodiment, the invention concerns the antibody or antigen binding fragment, wherein said radioactive isotope is an alpha, beta or positron emitting radionuclide.

According to an embodiment, the invention concerns the antibody or antigen binding fragment, wherein said alpha emitting radionuclide is selected among ²⁰⁹Bi, ²¹¹Bi, ²¹²Bi, ²¹³Bi, ²¹⁰Po, ²¹¹Po, ²¹²Po, ²¹⁴Po, ²¹⁵Po, ²¹⁶Po, ²¹⁸Po, ²¹¹At, ²¹⁵At, ²¹⁷At, ²¹⁸At, ²¹⁸Rn, ²¹⁹Rn, ²²⁰Rn, ²²²Rn, ²²⁶Rn, ²²¹Fr, ²²³Ra, ²²⁴Ra, ²²⁶Ra, ²²⁵Ac, ²²⁷Ac, ²²⁷Th, ²²⁸Th, ²²⁹Th, ²³⁰Th, ²³²Th, ²³¹Pa, ²³³U, ²³⁴U, ²³⁵U, ²³⁶U, ²³⁸U, ²³⁷Np, ²³⁸Pu, ²³⁹Pu, ²⁴⁰Pu, ²⁴⁴Pu, ²⁴¹Am, ²⁴⁴Cm, ²⁴⁵Cm, ²⁴⁸Cm, ²⁴⁹Cf, and ²⁵²Cf.

According to an embodiment, the invention concerns the antibody or antigen binding fragment, comprising a structure selected among IgG, IgG1, IgG2, IgG3, and IgG4.

According to an embodiment, the invention concerns the antibody or antigen binding fragment, comprising a structure selected among IgG, IgM, IgA, IgD, and IgE.

According to an embodiment, the invention concerns a self-assembly disassembly (SADA) polypeptide, wherein said polypeptide is linked to an antibody or antigen binding fragment according to the invention.

According to an embodiment, the invention concerns the antibody or antigen binding fragment, wherein said antibody or antigen binding fragment thereof is a bispecific and/or trispecific binding antibody or antigen binding fragment thereof.

According to an embodiment, the invention concerns the bispecific and/or trispecific binding antibody or antigen binding fragment, wherein said antibody or antigen binding fragment comprises a sequence according to any of the sequences selected among SEQ ID No. 20-30.

According to an embodiment, the invention concerns the bispecific and/or trispecific binding antibody or antigen binding fragment, wherein said antibody or antigen binding fragment comprises a signal peptide.

According to an embodiment, the invention concerns the bispecific and/or trispecific binding antibody or antigen binding fragment, wherein said bispecific and/or trispecific binding antibody comprises a first antibody or antigen binding fragment thereof according to the invention for binding to a first antigen, and a second antibody or antigen binding fragment for binding to a second antigen.

According to an embodiment, the invention concerns the bispecific antibody or antigen binding fragment, wherein said first antigen is CD38.

According to an embodiment, the invention concerns the bispecific antibody or antigen binding fragment, wherein said second antigen is CD3.

According to an embodiment, the invention concerns the bispecific antibody or antigen binding fragment, wherein said antibody comprises a sequence according to sequence ID No. 53 and/or 54.

According to an embodiment, the invention concerns the bispecific antibody or antigen binding fragment, wherein said antibody comprises a mutation in the Fc region, wherein said mutation is N297A and/or K322A.

According to an embodiment, the invention concerns the antibody or antigen binding fragment, wherein said antibody comprises at least one linker or at least two linkers.

According to an embodiment, the invention concerns the antibody or antigen binding fragment, wherein said antibody or antigen binding fragment comprises a first linker.

According to an embodiment, the invention concerns the antibody or antigen binding fragment, wherein said first linker comprises a sequence according to sequence ID No. 47.

According to an embodiment, the invention concerns the antibody or antigen binding fragment, wherein said antibody or antigen binding fragment comprises a second linker.

According to an embodiment, the invention concerns the antibody or antigen binding fragment, wherein said second linker comprises a sequence according to sequence ID No. 55.

According to an embodiment, the invention concerns the antibody or antigen binding fragment, wherein said linker is selected among sequence ID No 47 and 55.

According to an embodiment, the invention concerns the antibody or antigen binding fragment, wherein said antibody is a scFv, and wherein said scFv is linked to a second scFv, and wherein said second scFv is capable of binding to DOTA and/or DTPA.

According to an embodiment, the invention concerns the antibody or antigen binding fragment, wherein said antibody is a first scFv, and wherein said first scFv is linked to a second scFv, and wherein said second scFv comprises the sequence of SEQ ID No. 45.

According to an embodiment, the invention concerns the antibody or antigen binding fragment, wherein said first scFv comprises a variable heavy region according to sequence ID No. 8 and/or a variable light region according to sequence ID No. 1.

According to an embodiment, the invention concerns the antibody or antigen binding fragment, wherein said first scFv comprises a variable heavy region according to sequence ID No. 39 and/or a variable light region according to sequence ID No. 37.

According to an embodiment, the invention concerns the antibody or antigen binding fragment, wherein said first scFv comprises a variable heavy region according to sequence ID No. 16 and/or a variable light region according to sequence ID No. 6.

According to an embodiment, the invention concerns the antibody or antigen binding fragment, wherein said first scFv comprises a variable heavy region according to sequence ID No. 40 and/or a variable light region according to sequence ID No. 38.

According to an embodiment, the invention concerns the antibody or antigen binding fragment, wherein said variable heavy region and said variable light region is in the orientation VH-VL from the N-terminal to the C-terminal.

According to an embodiment, the invention concerns the antibody or antigen binding fragment, wherein said variable heavy region and said variable light region is in the orientation VL-VH from the N-terminal to the C-terminal.

According to an embodiment, the invention concerns the antibody or antigen binding fragment, wherein said antibody comprises at least three linkers.

According to an embodiment, the invention concerns the antibody or antigen binding fragment, wherein said antibody or antigen binding fragment comprises a third linker.

According to an embodiment, the invention concerns the antibody or antigen binding fragment, wherein said third linker comprises a sequence according to sequence ID No. 48.

According to an embodiment, the invention concerns the antibody or antigen binding fragment, wherein said antibody or antigen binding fragment comprises a fourth linker.

According to an embodiment, the invention concerns the antibody or antigen binding fragment, wherein said fourth linker comprises a sequence according to sequence ID No. 49.

According to an embodiment, the invention concerns the antibody or antigen binding fragment, wherein said antibody or antigen binding fragment comprises a fifth linker.

According to an embodiment, the invention concerns the antibody or antigen binding fragment, wherein said fifth linker comprises a sequence according to sequence ID No. 47.

According to an embodiment, the invention concerns the antibody or antigen binding fragment, wherein said antibody or antigen binding fragment comprises a sixth linker.

According to an embodiment, the invention concerns the antibody or antigen binding fragment, wherein said sixth linker comprises a sequence according to sequence ID No. 55.

According to an embodiment, the invention concerns the antibody or antigen binding fragment, wherein said antibody comprises a sequence according to sequence ID No. 46.

According to an embodiment, the invention concerns the antibody or antigen binding fragment, wherein said antibody or antigen binding fragment comprises a His tag, and wherein said his tag comprises the sequences according to sequence ID No. 50.

According to an embodiment, the invention concerns the antibody or antigen binding fragment, wherein said second antibody or antigen binding fragment thereof binds to DOTA and/or DTPA.

According to an embodiment, the invention concerns the antibody or antigen binding fragment, wherein said antibody or antigen binding fragment is linked to a self-assembly disassembly (SADA) polypeptide.

According to an embodiment, the invention concerns the antibody or antigen binding fragment, wherein said self-assembly disassembly (SADA) polypeptide has an amino acid sequence that shows at least 75% identity with that of a human homo-multimerizing polypeptide and being characterized by one or more multimerization dissociation constants (KD).

According to an embodiment, the invention concerns a polypeptide conjugate comprising a self-assembly disassembly (SADA) polypeptide according to the invention, and an antibody or antigen binding fragment according to the invention.

According to an embodiment, the invention concerns a polypeptide conjugate comprising a self-assembly disassembly (SADA) polypeptide, and wherein said conjugate further comprises the bispecific antibody according to the invention, wherein said first antigen is CD38 and wherein said second antigen is DOTA.

According to an embodiment, the invention concerns a polypeptide conjugate comprising a self-assembly disassembly (SADA) polypeptide, and at least a first binding domain that binds to a first target and is covalently linked to the SADA polypeptide.

According to an embodiment, the invention concerns the polypeptide conjugate, wherein said self-assembly disassembly (SADA) polypeptide has an amino acid sequence that shows at least 75% identity with that of a human homo-multimerizing polypeptide and being characterized by one or more multimerization dissociation constants (KD); and wherein said conjugate is being constructed and arranged so that it adopts a first multimerization state and one or more higher-order multimerization states, wherein: the first multimerization state is less than about −70 kDa in size, at least one of the higher-order multimerization states is a homo-tetramer or higher-order homo multimer greater than 150 kDa in size, wherein the higher-order homo-multimerized conjugate is stable in aqueous solution when the conjugate is present at a concentration above the SADA polypeptide KD, and the conjugate transitions from the higher-order multimerization state(s) to the first multimerization state under physiological conditions when the concentration of the conjugate is below the SADA polypeptide KD.

According to an embodiment, the invention concerns the polypeptide conjugate, wherein said conjugate comprises a chelator.

According to an embodiment, the invention concerns the conjugate, wherein said chelator comprises a metal ion.

According to an embodiment, the invention concerns the conjugate, wherein the metal ion is a radionuclide.

According to an embodiment, the invention concerns an isolated nucleic acid molecule encoding the antibody or antigen binding fragment according to the invention.

According to an embodiment, the invention concerns a n isolated nucleic acid molecule encoding an antibody or antigen binding fragment according to the invention.

According to an embodiment, the invention concerns a recombinant vector comprising the isolated nucleic acid molecule according to the invention.

According to an embodiment, the invention concerns a host cell comprising the recombinant vector according to the invention.

According to an embodiment, the invention concerns a method for the production of an antibody or antigen binding fragment thereof according to the invention comprising a step of culturing the host cell according to the invention in a culture medium under conditions allowing the expression of the antibody or fragment and separating the antibody or fragment from the culture medium.

According to an embodiment, the invention concerns a chimeric antigen receptor (CAR) comprising an antibody or antigen binding fragment according to the invention.

According to an embodiment, the invention concerns a CAR-T cell expressing a CAR according to the invention.

According to an embodiment, the invention concerns a population of CAR-T cells.

According to an embodiment, the invention concerns a composition comprising the population of CAR-T cells according to the invention.

According to an embodiment, the invention concerns a CAR-NK cell expressing a CAR according to the invention.

According to an embodiment, the invention concerns a population of CAR-NK cells.

According to an embodiment, the invention concerns a composition comprising the population of CAR-NK cells according to the invention.

According to an embodiment, the invention concerns a pharmaceutical composition comprising the antibody or antigen binding fragment according to the invention.

According to an embodiment, the invention concerns a T cell armed with the antibody or antigen binding fragment according to the invention.

According to an embodiment, the invention concerns a method of treating, preventing, alleviating and/or diagnosing the symptoms of a medical condition in a subject, comprising a step of administration of an antibody, an antigen binding fragment, a bispecific antibody, a trispecific antibody, a polypeptide conjugate, a composition and/or a CAR to a subject, and wherein said medical condition is characterized by expression of CD38 antigen.

According to an embodiment, the invention concerns the method, wherein said antibody, antigen binding fragment, bispecific antibody, trispecific antibody, polypeptide conjugate, composition and/or CAR is the antibody, antigen binding fragment, bispecific antibody, trispecific antibody, polypeptide conjugate, composition and/or CAR according to the invention.

According to an embodiment, the invention concerns use of the composition according to the invention in the manufacturing of a medicament for the treatment of a cancer, for use in a method according to the invention.

According to an embodiment, the invention concerns use of the antibody or antigen binding fragment according to the invention in the manufacturing of a medicament for the treatment of a cancer and/or for use in a method according to the invention.

According to an embodiment, the invention concerns a n in vitro use of an antibody or antigen binding fragment thereof according to the invention.

According to an embodiment, the invention concerns the method, wherein said medical condition is a cancer.

According to an embodiment, the invention concerns the method, wherein said cancer and/or said tumor is a metastasis.

According to an embodiment, the invention concerns the method, wherein said cancer cells is from an adult cancer.

According to an embodiment, the invention concerns the method, wherein said cancer cells is from a pediatric tumor.

According to an embodiment, the invention concerns the method, wherein said cancer cells and/or metastasis is a solid tumor.

According to an embodiment, the invention concerns the method, wherein said cancer cells and/or metastasis is selected among a multiple myeloma (MM), an AL amyloidosis, an Acute myeloid leukemia (AML), a myelodysplastic syndrome (MDS), a proliferative glomerulonephritis with monoclonal immune deposit, a C3 glomerulopathy associated with monoclonal gammopathy, a squamous cell carcinoma, a colon cancer, a non-small cell lung cancer, a mantle cell lymphoma, a diffuse large B-cell lymphoma, a follicular lymphoma, a NK-/T-cell lymphoma, a B-cell non-Hodgkin lymphoma, a T-cell acute lymphoblastic leukemia, a B-cell acute lymphoblastic leukemia, a chronic lymphocytic leukemia (CLL), a Waldenström macroglobulinemia, a solid cancer, a cancer with immunomodulatory activity and a cancer expressing CD38.

According to an embodiment, the invention concerns the method, wherein said medical condition is an autoimmune disease.

According to an embodiment, the invention concerns the method, wherein said autoimmune disease is selected among rheumatoid arthritis, multiple sclerosis, paraneoplastic syndromes, systemic lupus erythematosus, type 2 diabetes, Pemphigus Vulgaris, autoimmune hemolytic anemia, allergy and graft-versus-host disease.

In order to provide a clear and consistent understanding of the specification and claims, including the scope to be given such terms, the following definitions are provided.

Affinity: As is known in the art, “affinity” is a measure of the tightness with which a particular ligand (e.g., an antibody) binds to its partner (e.g., an epitope). Affinities can be measured in different ways.

Antibody: The term “antibody” is art-recognized terminology and is intended to include molecules or active fragments of molecules that bind to known antigens. Examples of active fragments of molecules that bind to known antigens include Fab and F(ab′)2 fragments. These active fragments can be derived from an antibody of the present invention by a number of techniques. For example, purified monoclonal antibodies can be cleaved with an enzyme, such as pepsin, and subjected to HPLC gel filtration. The appropriate fraction containing Fab fragments can then be collected and concentrated by membrane filtration and the like. The term “antibody” also includes bispecific and chimeric antibodies and other available formats.

Antibody fragment: An antibody fragment is a portion of an antibody such as F(ab′)2, F(ab)2, Fab′, Fab, Fv, sFv and the like. Regardless of structure, an antibody fragment binds with the same antigen that is recognized by the intact antibody. For example, an 3F8 monoclonal antibody fragment binds with an epitope recognized by 3F8. The term “antibody fragment” also includes any synthetic or genetically engineered protein that acts like an antibody by binding to a specific antigen to form a complex. For example, antibody fragments include isolated fragments consisting of the variable regions, such as the “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.

Bispecific antibody: A bispecific antibody is an antibody that can bind simultaneously to two targets which are of different structure. Bispecific antibodies (bsAb) and bispecific antibody fragments (bsFab) have at least one arm that specifically binds to an antigen, for example, GD2 and at least one other arm that specifically binds to another antigen, for example a targetable conjugate that bears a therapeutic or diagnostic agent. A variety of bispecific fusion proteins can be produced using molecular engineering. In one form, the bispecific fusion protein is divalent, consisting of, for example, a scFv with a single binding site for one antigen and a Fab fragment with a single binding site for a second antigen. In another form, the bispecific fusion protein is tetravalent, consisting of, for example, an IgG with two binding sites for one antigen and two identical scFv for a second antigen.

Chimeric antibody: A chimeric antibody is a recombinant protein that contains the variable domains including the complementarity-determining regions (CDRs) of an antibody derived from one species, for example a rodent antibody, while the constant domains of the antibody molecule is derived from those of a human antibody. The constant domains of the chimeric antibody may also be derived from that of other species, such as a cat or dog.

Effective amount: As used herein, the term “effective amount” refers to an amount of a given compound, conjugate or composition that is necessary or sufficient to realize a desired biologic effect. An effective amount of a given compound, conjugate or composition in accordance with the methods of the present invention would be the amount that achieves this selected result, and such an amount can be determined as a matter of routine by a person skilled in the art, without the need for undue experimentation.

Humanized antibody: A humanized antibody is a recombinant protein in which the CDRs from an antibody from one species; e.g., a rodent antibody, is transferred from the heavy and light variable chains of the rodent antibody into human heavy and light variable domains. The constant domain of the antibody molecule is derived from those of a human antibody.

A human antibody may be an antibody obtained from transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas.

Prevent: As used herein, the terms “prevent”, “preventing” and “prevention” refer to the prevention of the recurrence or onset of one or more symptoms of a disorder in a subject as result of the administration of a prophylactic or therapeutic agent.

Radioactive isotope: Examples of radioactive isotopes that can be conjugated to antibodies for use diagnostically or therapeutically include, but are not limited to, ²¹¹At, ¹⁴C, ⁵¹Cr, ⁵⁷Co, ⁵⁸Co, ⁶⁷Cu, ¹⁵²Eu, ⁶⁷Ga, ³H, ¹¹¹In, ⁵⁹Fe, ²¹²Pb, ¹⁷⁷Lu, ³²P, ²²³Ra, ²²⁴Ra, ¹⁸⁶Re, ¹⁸⁸Re, ⁷⁸Se, ³⁵S, ^{99m}Tc, ²²⁷Th, ⁸⁹Zr, ⁹⁹Y, ¹²³I, ¹²⁴I, ¹²⁵I, ¹³¹I, ^{94m}Tc, ⁶⁴Cu, ⁶⁸Ga, ⁶⁶Ga, ⁷⁶Br, ⁸⁶Y, ⁸²Rb, ^{110m}In, ¹³N, ¹¹C, ¹⁸F and alpha-emitting particles. Non-limiting examples of alpha-emitting particles include ²⁰⁹Bi, ²¹¹Bi, ²¹²Bi, ²¹³Bi, ²¹⁰Po, ²¹¹Po, ²¹²Po, ²¹⁴Po, ²¹⁵Po, ²¹⁶Po, ²¹⁸Po, ²¹¹At, ²¹⁵At, ²¹⁷At, ²¹⁸At, ²¹⁸Rn, ²¹⁹Rn, ²²⁰Rn, ²²²Rn, ²²⁶Rn, ²²¹Fr, ²²³Ra, ²²⁴Ra, ²²⁶Ra, ²²⁵Ac, ²²⁷Ac, ²²⁷Th, ²²⁸Th, ²²⁹Th, ²³⁰Th, ²³²Th, ²³¹Pa, ²³³U, ²³⁴U, ²³⁵U, ²³⁶U, ²³⁸U, ²³⁷Np, ²³⁸Pu, ²³⁹Pu, ²⁴⁰Pu, ²⁴⁴Pu, ²⁴¹Am, ²⁴⁴Cm, ²⁴⁵Cm, ²⁴⁸Cm, ²⁴⁹Cf, and ²⁵²Cf.

Subject: By “subject” or “individual” or “animal” or “patient” or “mammal,” is meant any subject, particularly a mammalian subject, for whom diagnosis, prognosis, or therapy is desired. Mammalian subjects include humans and other primates, domestic animals, farm animals, and zoo, sports, or pet animals such as dogs, cats, guinea pigs, rabbits, rats, mice, horses, cattle, cows, and the like.

Treatment: As used herein, the terms “treatment”, “treat”, “treated” or “treating” refer to prophylaxis and/or therapy, particularly wherein the object is to prevent or slow down (lessen) an undesired physiological change or disorder, such as the progression of multiple sclerosis. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The following terms are used interchangeable to refer to the same constructs: 8DC1 and 8DC-1; 8DC2 and 8DC-2; 8DC3 and 8DC-3; 8DC4 and 8DC-4; 8DC5 and 8DC-5.

### FIGURES

FIG. 1A-1D shows the purity of the T-cell BsAbs assessed using HPLC technique

FIG. 2A-B shows flow cytometry, with CD38(+) cancer cells (RPMI, Raji, and Daudi) stained with different concentrations of the bispecific antibodies followed by detection of the antibody using a goat-anti-human IgG fluorochrome-conjugated secondary antibody.

FIG. 3A-3D shows DRTOK, 8DC1OK and 8DC3OK potently redirect T-cells to lyse CD38(+) tumor cells.

FIG. 4A-4C shows the purity of the radioimmunotherapy antibodies assessed using HPLC technique.

FIG. 5 shows the thermal stability of the BsAbs tested at 40° C. over time using HPLC method.

FIG. 6A-6D shows SPR experiments: the humanized 8DC3 antibody retained most of its affinity after humanization.

FIG. 7A-7B shows the mean fluorescence intensity (MFI) of the BsAbs binding to tumor cells.

FIG. 8A-8B shows the purity of 8DC-4 and 8DC-5 assessed using H PLC-SEC method.

FIG. 9 shows thermal stability of 8DC-4 at 40° C. over time assessed using HPLC method.

FIG. 10A-10B shows affinity (SPR) data for 8DC-4 and 8DC-5.

FIG. 11A-11B shows thermal denaturation temperature (Tm) of 8DC-3, 8DC-4 and 8DC-5 assessed by thermal shift assay assesses.

FIG. 12A-12B shows internalization data for 8DC-3 and 8DC-4.

All cited references are incorporated by reference.

The accompanying Figures and Examples are provided to explain rather than limit the present invention. It will be clear to the person skilled in the art that aspects, embodiments, claims and any items of the present invention may be combined.

Unless otherwise mentioned, all percentages are in weight/weight. Unless otherwise mentioned, all measurements are conducted under standard conditions (ambient temperature and pressure). Unless otherwise mentioned, test conditions are according to European Pharmacopoeia 8.0.

### EXAMPLES

### Example 1: IgG Format

Humanization and sequence design process: The murine variable regions (VL and VH) of AT13/5 anti-CD38 antibody were humanized by CDR grafting method using the following human germline templates: VL (IGKV1-NL1*01 AND IGKJ4*01), VH (IGHV4-4*08 AND IGHJ5*01). Four humanized VL and 7 humanized VH variants were designed SEQ ID No. 2-5 and 9-15 respectively. The VL and VH of daratumumab antibody is also shown in SEQ ID NO. 7 and SEQ ID NO 17. All VL and VH combinations were used to generate 28 different humanized IgG antibodies. The yield and purity (measured by HPLC) of the humanized IgG variants is shown in Table 1.

TABLE 1
Yield and purity of the humanized IgG variants.

| Protein Name | Titer (mg/L) | HPLC peak % |
|---|---|---|
| Chimeric | 519.16 | 100.00 |
| L1H1 | 480.12 | 97.02 |
| L1H2 | 511.03 | 97.46 |
| L1H3 | 524.47 | 96.48 |
| L1H4 | 502.15 | 95.52 |
| L1H5 | 487.25 | 97.53 |
| L1H6 | 496.20 | 96.67 |
| L1H7 | 541.62 | 98.33 |
| L2H1 | 449.61 | 97.56 |
| L2H2 | 527.65 | 97.94 |
| L2H3 | 540.64 | 85.28 |
| L2H4 | 336.86 | 53.79 |
| L2H5 | 503.83 | 83.98 |
| L2H6 | 516.53 | 97.52 |
| L2H7 | 738.63 | 98.14 |
| L3H1 | 813.13 | 97.28 |
| L3H2 | 622.18 | 97.48 |
| L3H3 | 790.12 | 97.27 |
| L3H4 | 639.94 | 96.36 |
| L3H5 | 558.97 | 98.04 |
| L3H6 | 607.22 | 97.49 |
| L3H7 | 598.78 | 100.00 |
| L4H1 | 461.33 | 97.72 |
| L4H2 | 376.88 | 96.96 |
| L4H3 | 439.52 | 95.88 |
| L4H4 | 453.56 | 94.07 |
| L4H5 | 439.20 | 96.77 |
| L4H6 | 404.40 | 95.44 |
| L4H7 | 425.97 | 97.98 |

Surface Plasmon Resonance (SPR): To measure the binding affinity of the humanized IgG antibodies to their target antigen, an anti-human Fc surface was generated on a HC30M chip by amine coupling. The humanized IgG antibodies were diluted to either 10 μg/ml or 5 μg/ml and printed onto the anti-human Fc surface. Different dilutions of human CD38 antigen with a starting concentration of 1000 nM were prepared and flown over the surface. Association was observed for 5 minutes and dissociation was observed for 15 minutes. The binding kinetics of the humanized antibodies are shown in Table 2.

TABLE 2
The binding kinetics of the humanized antibodies.

| | CD38 SPR | | |
|---|---|---|---|
| Protein Name | ka | kd | KD |
| Chimeric | 7.7E+05 | 2.1E−03 | 2.8E−09 |
| L1H1 | 3.8E+04 | 3.3E−02 | 8.7E−07 |
| L1H2 | 3.7E+04 | 1.5E−02 | 4.0E−07 |
| L1H3 | 3.8E+04 | 3.1E−02 | 8.1E−07 |
| L1H4 | 2.7E+04 | 7.8E−03 | 3.0E−07 |
| L1H5 | 1.9E+04 | 3.4E−02 | 1.8E−06 |
| L1H6 | 1.7E+04 | 5.2E−04 | 3.1E−08 |
| L1H7 | 8.5E+03 | 7.2E−02 | 8.5E−06 |
| L2H1 | 3.9E+04 | 3.7E−02 | 9.5E−07 |
| L2H2 | 6.4E+04 | 5.8E−02 | 9.1E−07 |
| L2H3 | 3.1E+04 | 3.0E−02 | 8.3E−07 |
| L2H4 | 1.2E+04 | 1.7E−03 | 1.9E−07 |
| L2H5 | 1.0E+03 | 1.4E−02 | 2.9E−05 |
| L2H6 | 2.2E+04 | 4.3E−02 | 1.9E−06 |
| L2H7 | 8.4E+03 | 3.3E−03 | 3.9E−07 |
| L3H1 | 3.1E+04 | 2.1E−02 | 6.5E−07 |
| L3H2 | 2.9E+04 | 1.3E−02 | 4.6E−07 |
| L3H3 | 4.9E+04 | 5.8E−02 | 1.2E−06 |
| L3H4 | 4.3E+04 | 5.1E−02 | 1.2E−06 |
| L3H5 | 9.6E+03 | 3.6E−01 | 5.3E−05 |
| L3H6 | 3.7E+04 | 5.0E−02 | 1.3E−06 |
| L3H7 | 9.6E+03 | 9.4E−04 | 1.0E−07 |
| L4H1 | 8.8E+04 | 9.5E−03 | 1.0E−07 |
| L4H2 | 8.5E+04 | 2.1E−02 | 2.5E−07 |
| L4H3 | 9.6E+04 | 1.9E−02 | 2.0E−07 |
| L4H4 | 9.7E+04 | 1.4E−02 | 1.4E−07 |
| L4H5 | 2.8E+04 | 8.4E−03 | 3.2E−07 |
| L4H6 | 6.1E+04 | 3.6E−02 | 5.8E−07 |
| L4H7 | 3.0E+04 | 2.5E−02 | 8.7E−07 |

TABLE 3
Human germline content

| Candidate | % human germline content |
|---|---|
| chimeric VL | 73.70% |
| Hu anti-CD38 VL1 | 86.30% |
| Hu anti-CD38 VL2 | 88.40% |
| Hu anti-CD38 VL3 | 87.40% |
| Hu anti-CD38 VL4 | 87.40% |
| Hu anti-CD38 VL5 | 83.20% |
| Daratumumab VL | 100.00% |
| chimeric VH | 57.70% |
| Hu anti-CD38 VH1 | 83.50% |
| Hu anti-CD38 VH2 | 81.40% |
| Hu anti-CD38 VH3 | 83.50% |
| Hu anti-CD38 VH4 | 84.50% |
| Hu anti-CD38 VH5 | 84.50% |
| Hu anti-CD38 VH6 | 85.60% |
| Hu anti-CD38 VH7 | 84.50% |
| Hu anti-CD38 VH8 | 79.40% |
| Daratumumab VH | 94.90% |
| Hu anti-CD38 VL7 | 87.4% |
| Hu anti-CD38 VL8 | 83.2% |
| Hu anti-CD38 VH10 | 74.2% |
| Hu anti-CD38 VH11 | 78.4% |

### Example 2: Second Round of Humanization of Clone AT13/5

Due to the suboptimal binding of the humanized AT13/5 IgG constructs developed in Example 1, a new humanized construct was generated based on rational design principles using a computationally derived homology model of the Fv fragment of murine clone AT13/5. Computational modelling was done using Biovia Discovery Studio software (Dassault Systemes) and the homology model of AT13/5 Fv was constructed using the structures of the following protein data bank templates: 3RAJ, 5TH9, 4ZXB, 3KJ4, and 3U9P. The new humanized AT13/5 clone based on this additional modelling is found in VL5 (Seq. No. 6) and VH8 (Seq. No. 16). For comparison, a construct based on a previously published humanized AT13/5 from Ellis et al. [14] was generated and is represented in the sequences VL7 and VH10.

### Example 3: T-Cell Bispecific Format

Humanization and sequence design process: The new humanized VL5 and VH8 were used to generate CD3xCD38 bispecific antibodies using the IgG-L-scFv format. The sequences of these bispecific antibodies have SEQ ID NO. 20-23. A similar bispecific antibody (DRTOK) was generated using the Daratumumab VL and VH. Its sequences have SEQ ID NO. 24 and 25. The Fc region of the antibodies with SEQ ID Nos 20-25 contains N297A (to remove Fc glycosylation) and K322A (to remove complement binding).

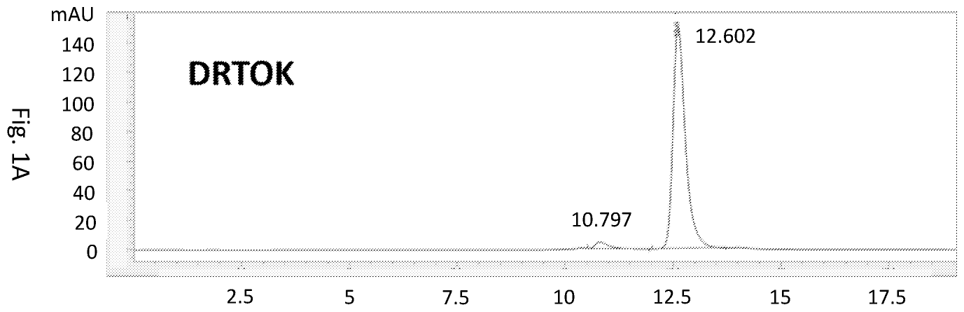

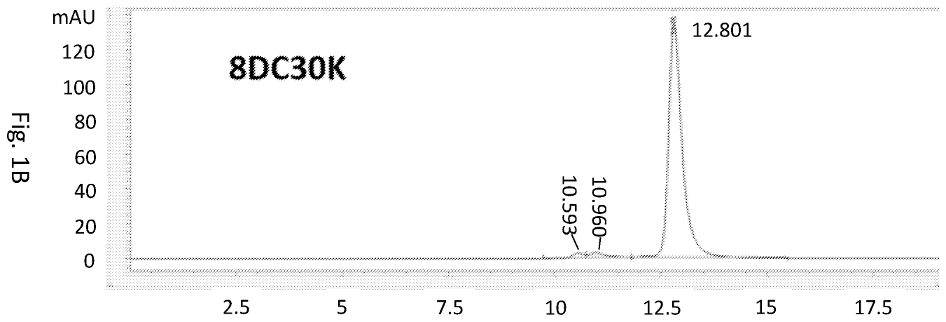

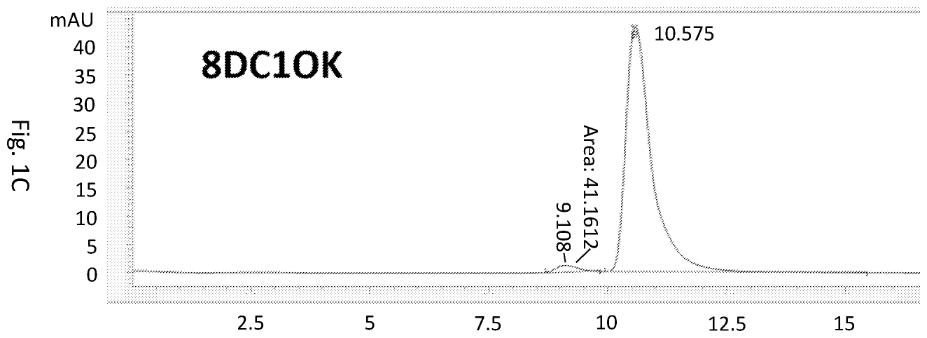

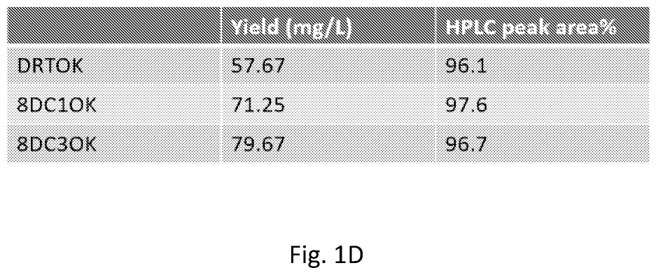

Protein generation and quality testing: Transient transfection of expi293F™ cells was used to generate the BsAbs, which were then purified by potein A resin (FIG. 1A-D). The purity of the T-cell BsAbs was assessed using Size Exclusion Chromatography HPLC technique (FIG. 1A-D). In addition, the thermal stability of the BsAbs was tested at 40° C. over time using Size Exclusion Chromatography HPLC method.

SPR: The binding of the BsAbs to their target antigen was tested using SPR technology. The BsAbs were captured on an anti-human Fc chip. Then different concentrations of human CD38 protein was flown over the chip followed by flowing the buffer only. The kinetic of BsAb binding to human CD38 is shown in table 4. The constructs 8DC1OK and 8DC3OK showed superior affinity to CD38 than DRTOK.

TABLE 4
CD38 TCB SPR data for human CD38

| | avg ka | SD ka | avg kd | SD kd | avg KD | SD KD | | |
|---|---|---|---|---|---|---|---|---|
| Antibody | (M⁻¹S⁻¹) | (M⁻¹S⁻¹) | (S⁻¹) | (S⁻¹) | (nM) | (nM) | N | Antibody |
| DRTOK | 9.1E+04 | 1.5E+04 | 3.4E−03 | 1.7E−04 | 38.8 | 5.7 | 6 | DRTOK |
| 1 μg/ml | | | | | | | | 1 μg/ml |
| 8DC3OK | 2.1E+05 | 4.1E+04 | 1.8E−03 | 1.6E−04 | 8.8 | 2.1 | 6 | 8DC3OK |
| 1 μg/ml | | | | | | | | 1 μg/ml |
| 8DC1OK | 4.7E+05 | 3.1E+04 | 7.6E−04 | 2.9E−05 | 1.6 | 0.1 | 6 | 8DC1OK |
| 1 μg/ml | | | | | | | | 1 μg/ml |

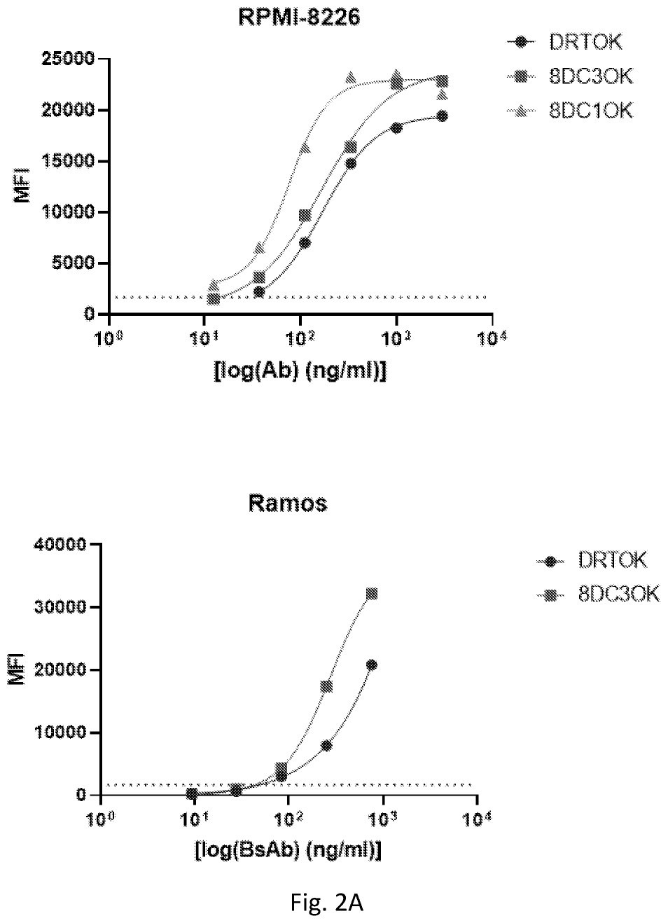

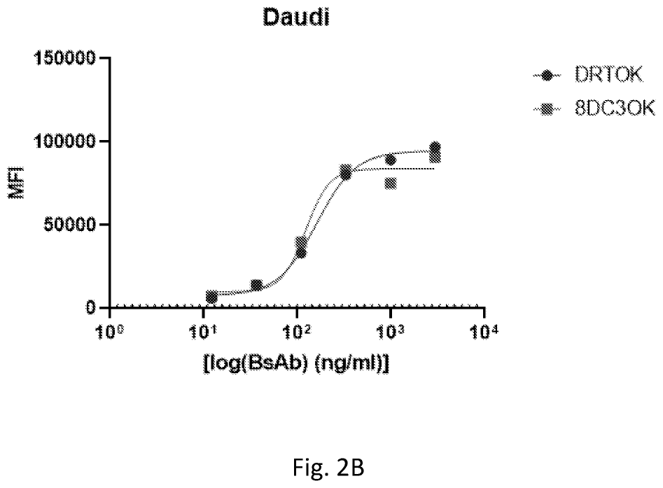

FACS: For flow cytometry, CD38(+) cancer cells (RPMI, Raji, and Daudi) were stained with different concentrations of the bispecific antibodies followed by detection of the antibody using a goat-anti-human IgG fluorochrome-conjugated secondary antibody. As shown in FIG. 2A-B, all BsAbs bind CD38(+) cancer cells. The EC50 of the humanized 8DC3OK is only two-fold weaker than the chimeric antibody 8DC1OK on RPM18226 cells (167 vs 76 ng/ml).

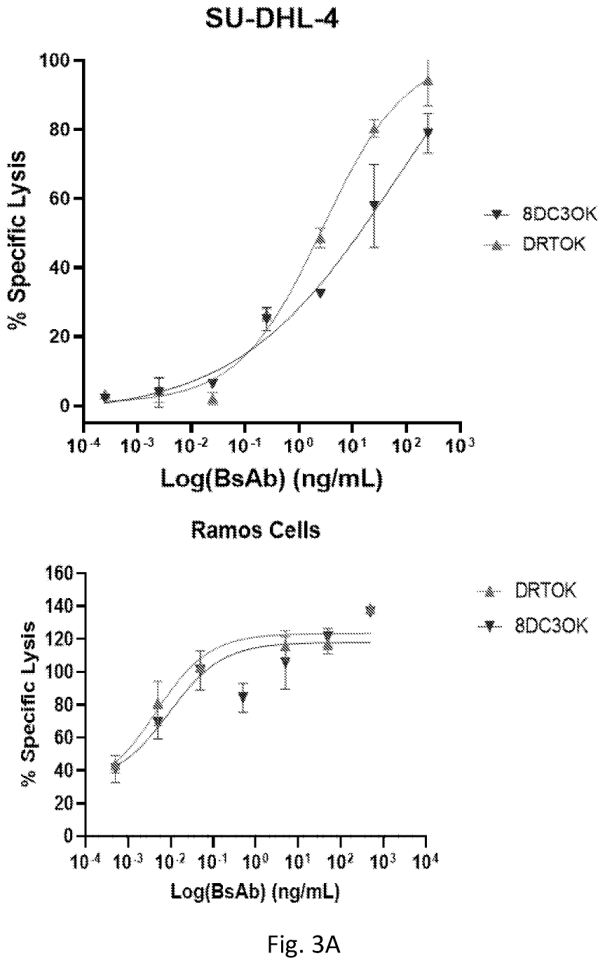

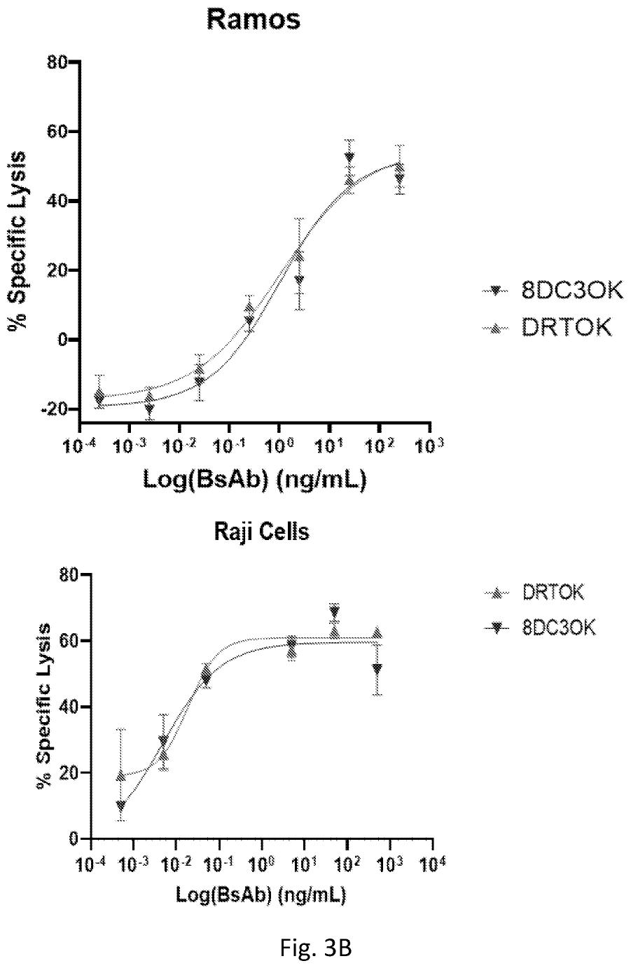

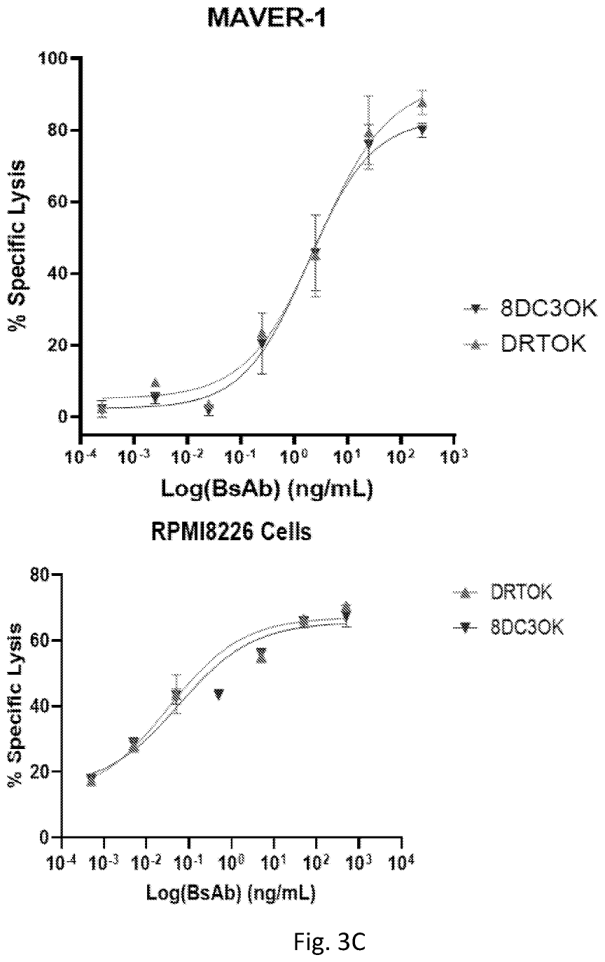

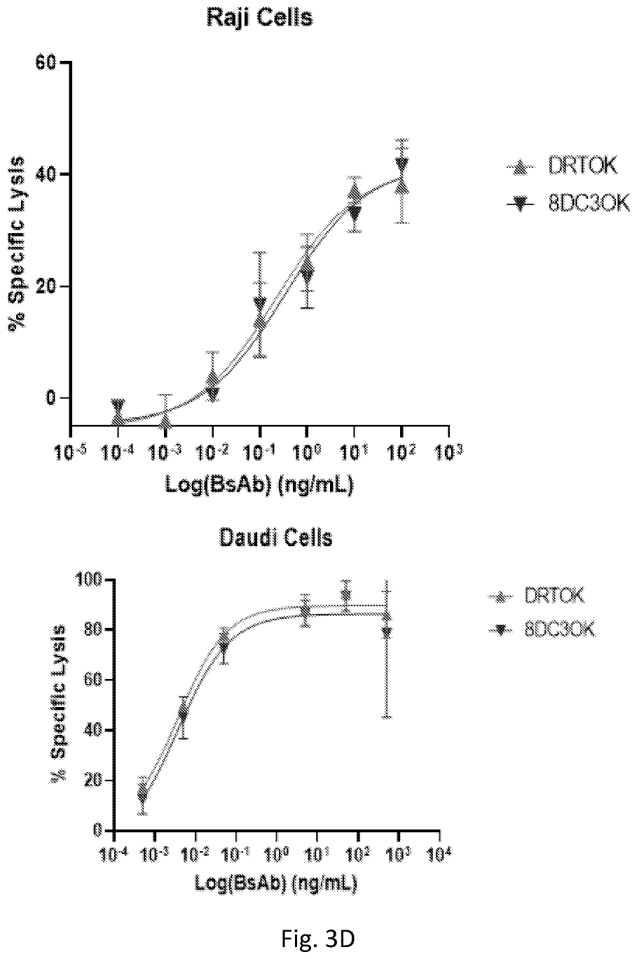

TDCC: To investigate the potency of the BsAbs in vitro, CD38(+) Raji cells were incubated with activated human T cells in the presence of different concentrations of BsAbs. As shown in FIG. 3A-D, DRTOK, 8DC1OK and 8DC3OK potently redirect T-cells to lyse CD38(+) tumor cells.

### Example 4: Radioimmunotherapy Agents

Humanization and sequence design process: The sequence of 5 antibodies for generation of radioimmunotherapy product is shown in sequence ID No. 26-30. An overview of the sequences in the constructs are provided in table 5. 8DC-1 contains the murine AT13/5 sequence. 8DC-2 contains the previously published huAt13/5 clone from Ellis et al. 8DC-3 contains VL5 and VH8 from the second humanization round. 8DC4 and 8DC5 are variations of 8DC3 where an extra disulfide has been engineered and the VL/VH chain orientation was varied.

TABLE 5
Sequences of construct 8DC1, 8DC2, 8DC3, 8DC4 and 8DC5

| | 8DC1 | 8DC2 | 8DC3 | 8DC4 | 8DC5 |
|---|---|---|---|---|---|
| VL | Murine | VL7 | VL5 | VL8 | VL8 |
| VH | Murine | VH10 | VH8 | VH11 | VH11 |

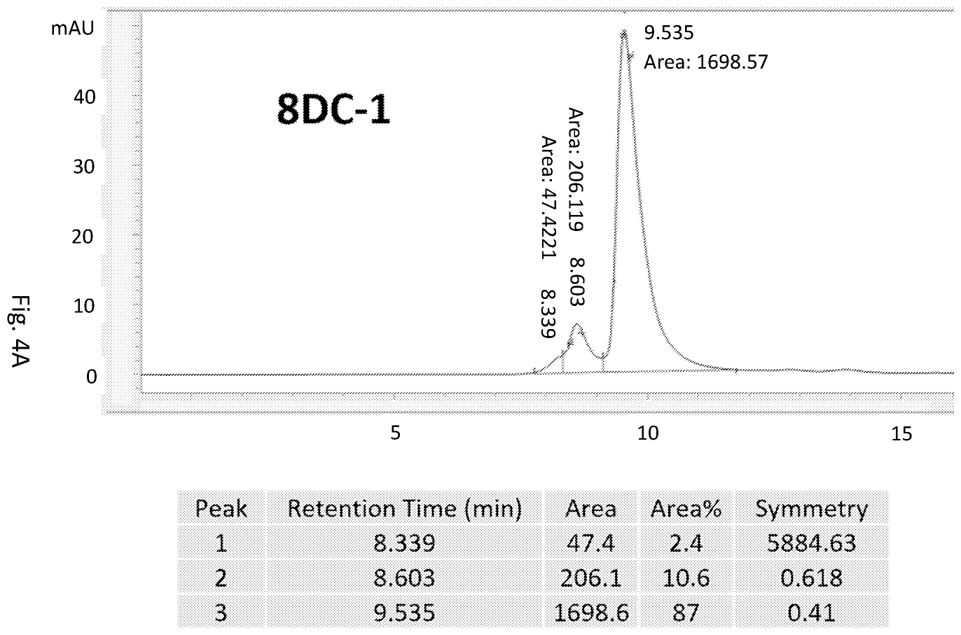

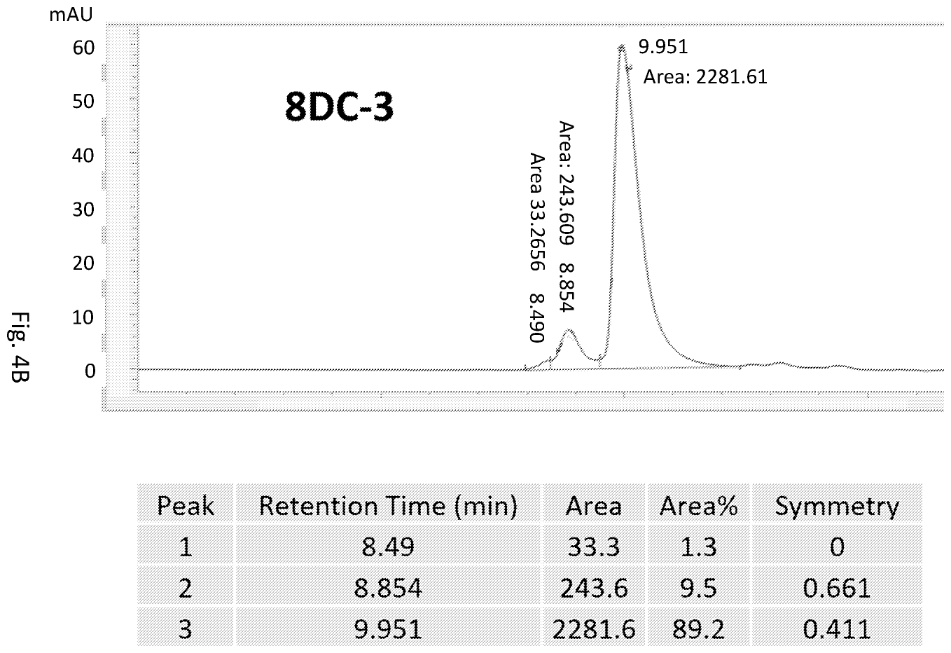

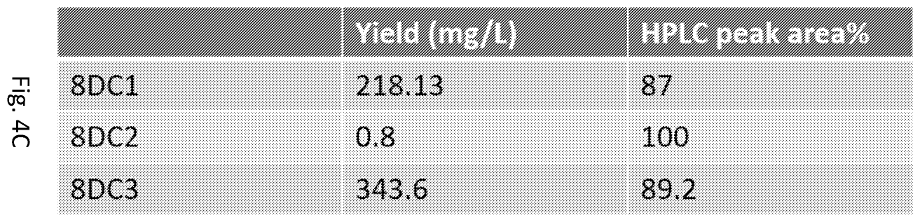

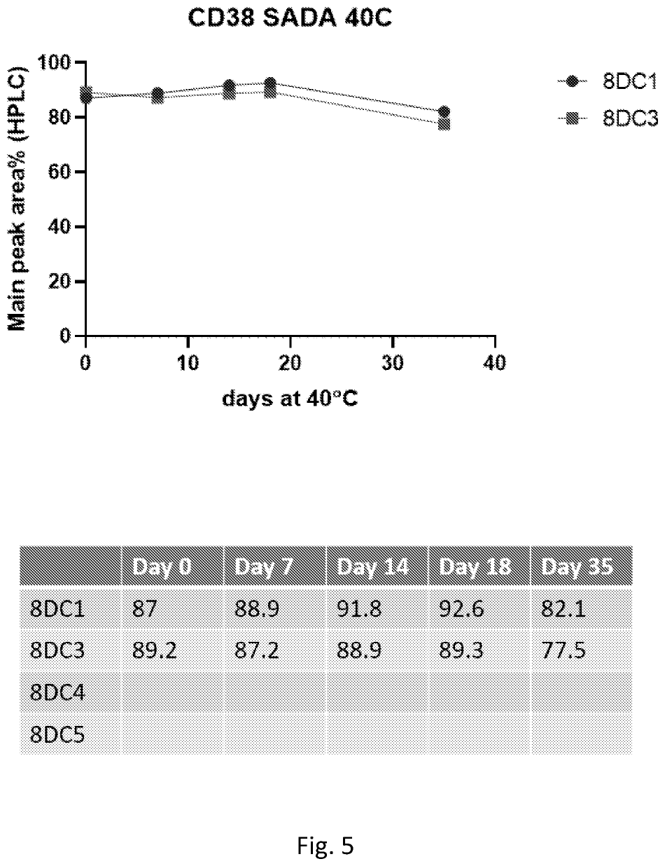

Protein generation and quality testing: Transient transfection of HEK SUS cells was performed to generate the SADA BsAbs and purification was done using protein-L resin (FIG. 25 4A-C). The purity of the radioimmunotherapy antibodies was assessed using HPLC-SEC technique (FIG. 4A-C). In addition, the thermal stability of the BsAbs was tested at 40° C. over time using HPLC method (FIG. 5).

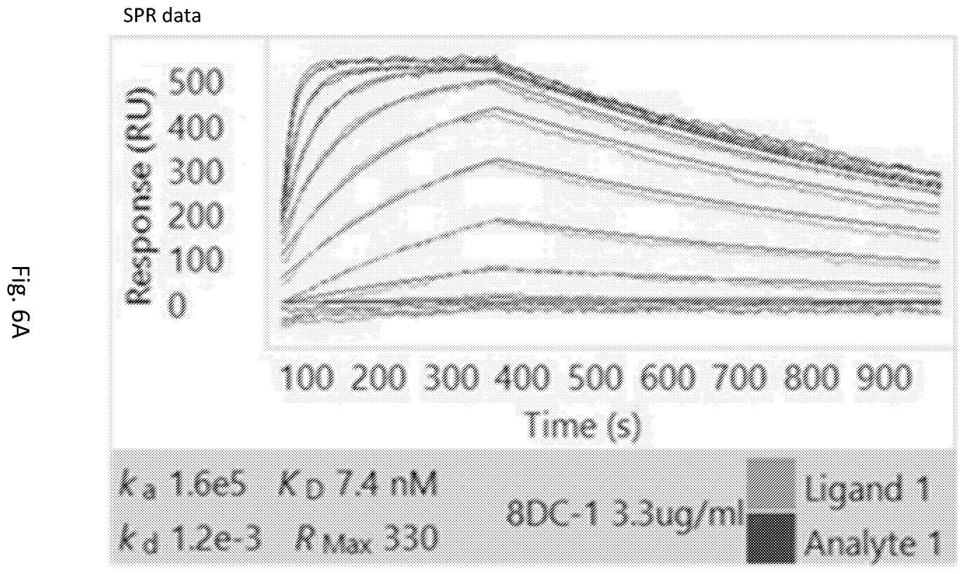

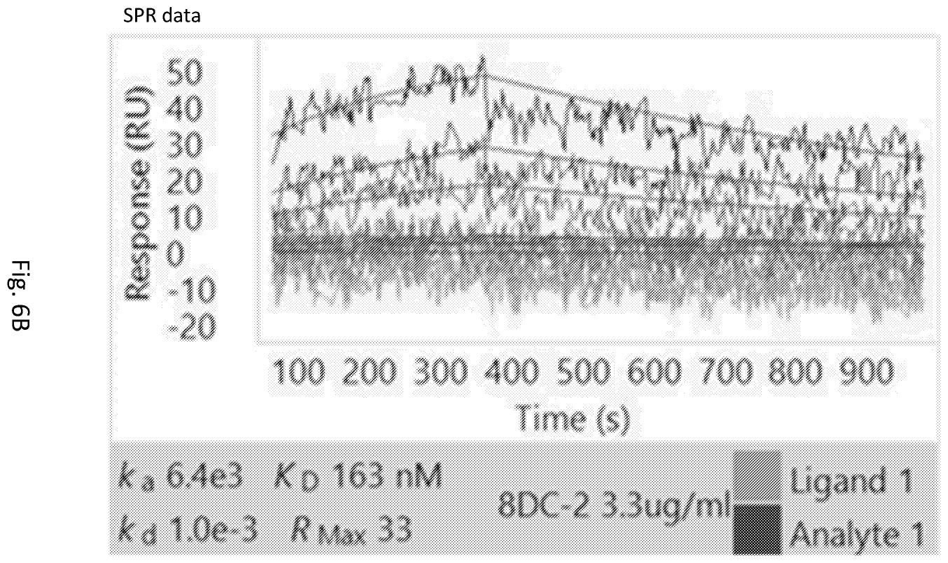

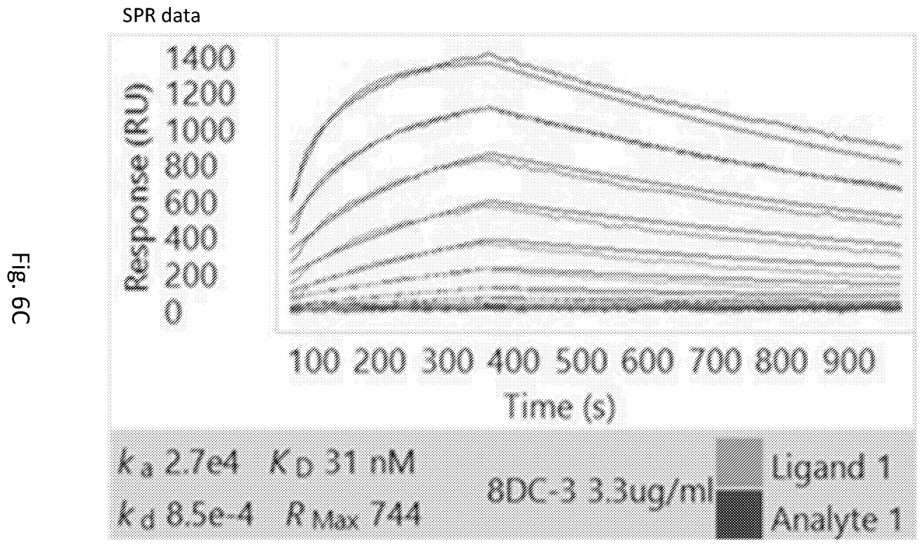

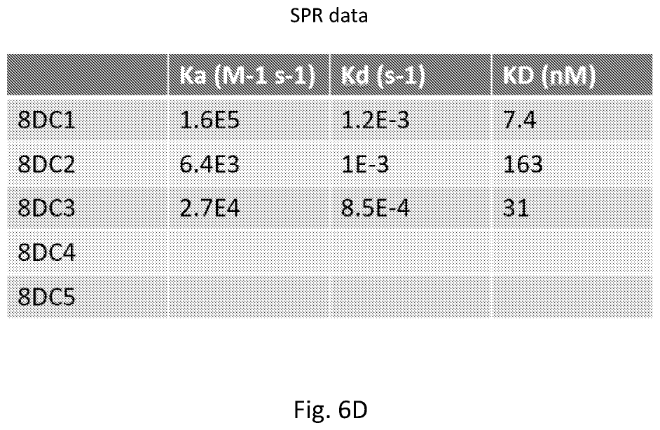

SPR: For SPR experiments, protein-L was immobilized on an HC30M chip. The antibodies were captured on the Protein-L chip followed by flowing different concentrations of human CD38 antigen over the chip (association phase). Then buffer was flown over the chip and antigen dissociation was measured (dissociation phase). As shown in (FIG. 6A-D], the humanized 8DC3 antibody retained most of its affinity after humanization (KD 31 nM compared to the KD of the murine antibody 7.4 nM).

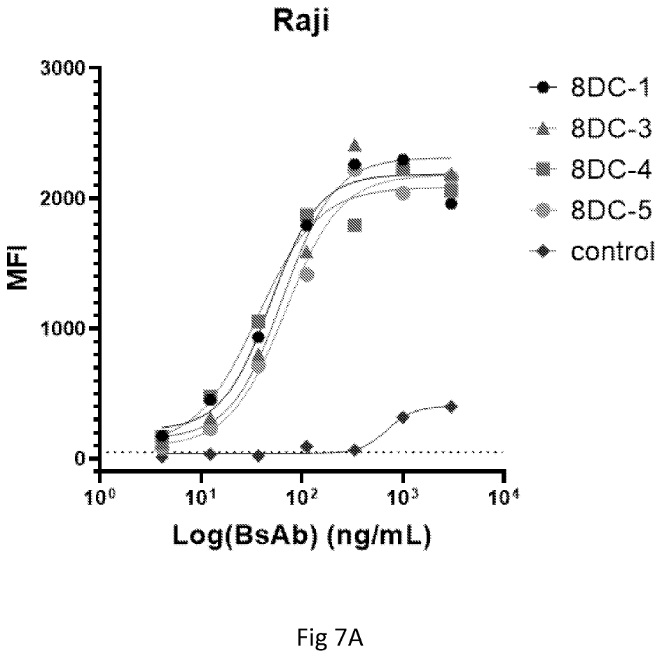

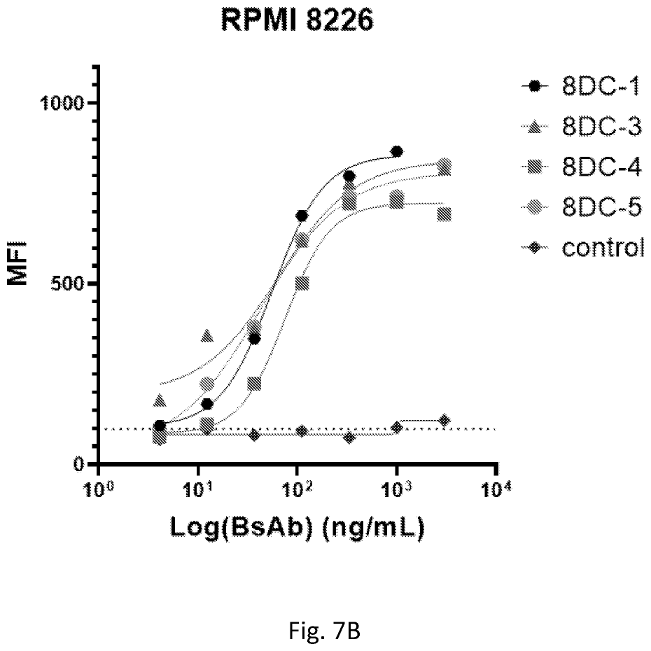

FACS: For flow cytometry assay, CD38(+) RPM18226 and Raji cells were stained with the BsAbs for 30 min followed by washing and staining with an anti-his tag antibody. FIG. 7A-B shows the mean fluorescence intensity (MFI) of the BsAbs binding to tumor cells.

There are several features that makes the 8DC3 construct superior to the published humanized sequence used to make the 8DC2 antibody: Stronger binding affinity of 8DC3 (KD=31 nM) versus 8DC2 (KD=163 nM) to CD38 (FIG. 6B-C), higher antibody yields: 8DC3 (343.6 mg/L), 8DC2 (0.8 mg/L) (FIG. 4C), which is indicative of higher intrinsic stability for 8DC3.

### Example 5: Radioimmunotherapy Agents

8DC3, 8DC4 and 8DC5 described in Example 4 were chosen for further experiments.

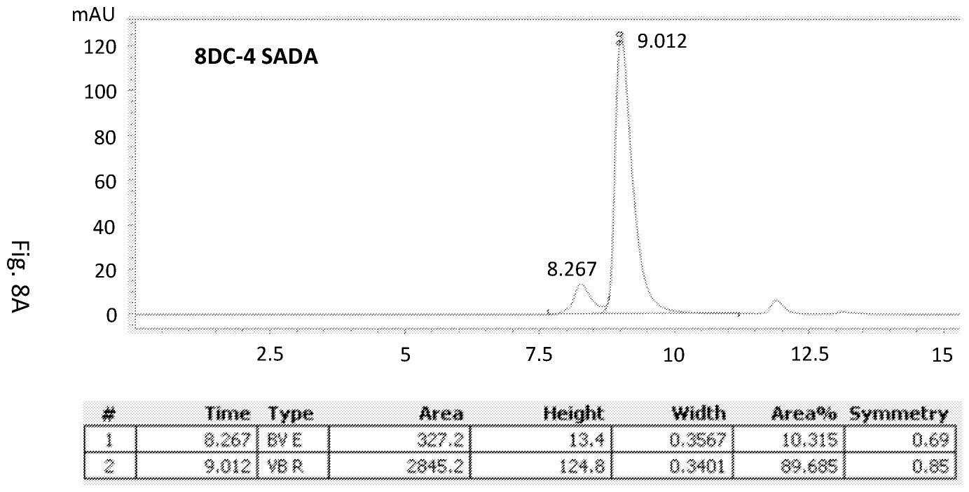

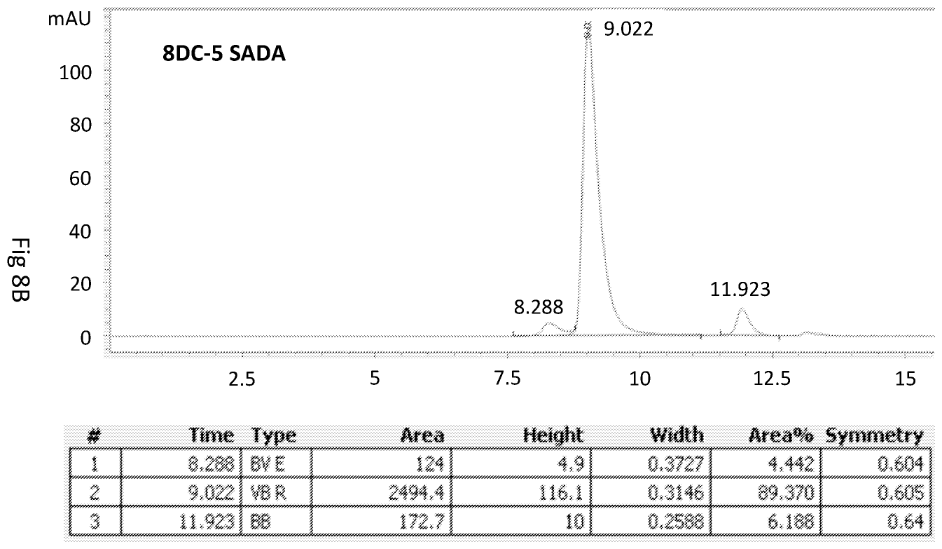

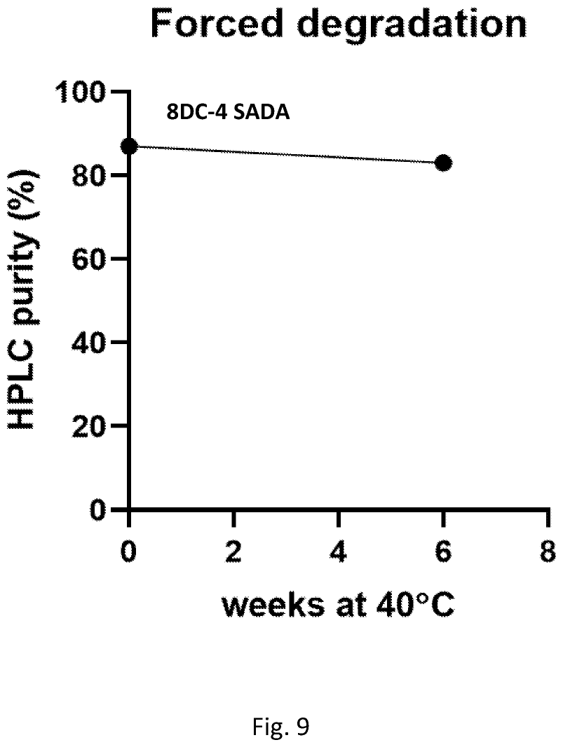

Protein generation and quality testing: Transfection of expiCHO cells was performed to generate 8DC4 and 8DC5 and purification was done using protein-L resin. The purity of 8DC4 and 8DC5 was assessed using HPLC-SEC technique (FIG. 8A-8B). In addition, the thermal stability of 8DC4 was tested at 40° C. over time using HPLC method (FIG. 9). 8DC3 was produced as described in Example 4.

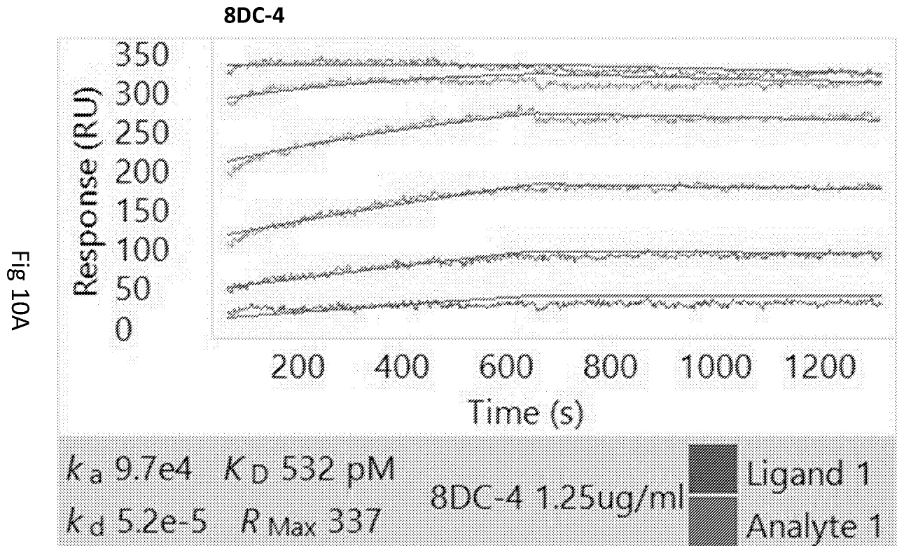

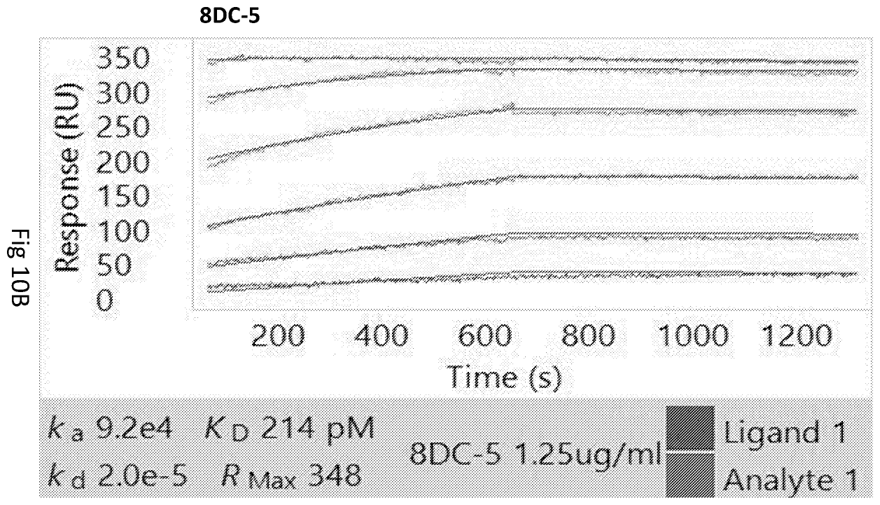

SPR: For the SPR experiment, a biosensor NiHC200M Chip was used to capture the His-tagged SADA molecules. Afterwards, recombinant human CD38 protein was injected over the chip and the binding kinetics of the CD38 protein and 8DC4 or 8DC5 was measured. As shown in FIG. 10A-10B and Table 6 below, both constructs showed favorable binding kinetics.

TABLE 6
Affinity (SPR) data for 8DC4 and 8DC5

| | AVG kₐ (M⁻¹S⁻¹) | AVG k_{d} (M⁻¹) | AVG K_{D} (M) |
|---|---|---|---|
| 8DC4 | 8.9E+04 | 2.6E−05 | 2.8E−10 |
| 8DC5 | 9.6E+04 | 1.6E−05 | 1.7E−10 |

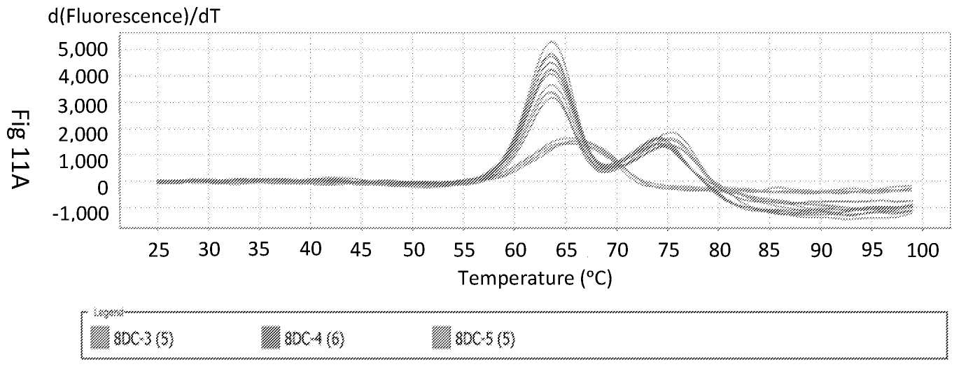

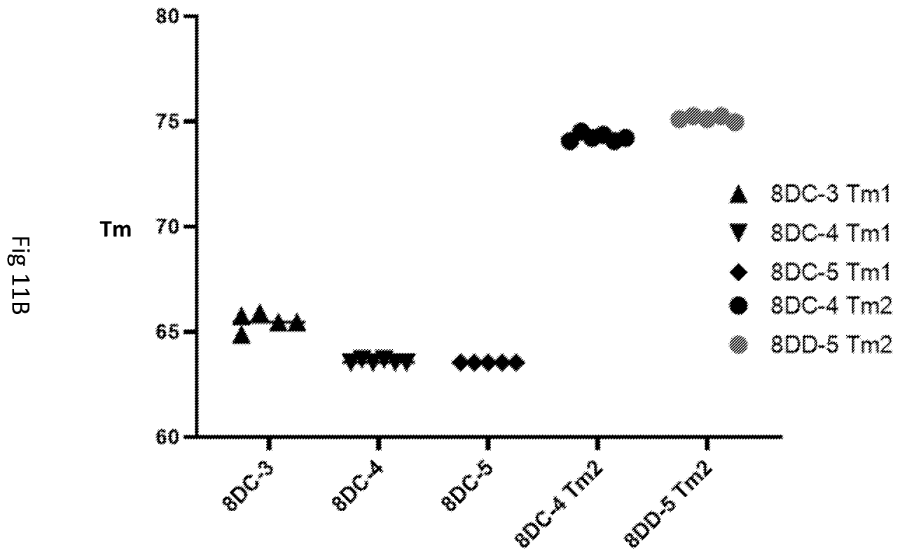

Thermal shift assay: Thermal shift assay assesses the stability of proteins by measuring their thermal denaturation temperature (Tm). In this assay, 8DC3, 8DC4 and 8DC5 were mixed with a dye that fluoresce in the absence of water. Then, the mixture was heated in a PCR machine. When the protein starts to denature, the hydrophobic surfaces become exposed and bind to the dye, which then fluoresce. As shown in FIG. 11A-11B and Table 7, the 8DC3 construct has a single Tm while both 8DC4 and 8DC5 that contain an extra disulfide bond in their a nti-CD38 scFv have two Tm peaks. The second Tm (Tm2) of 8DC4 and 8DC5 molecules are significantly higher than the Tm of 8DC3 suggesting an increase in SADA protein stability by incorporation of extra disulfide bonds.

TABLE 7
Tm for 8DC3, 8DC4 and 8DC5

| | 8DC3 | 8DC4 | 8DC5 |
|---|---|---|---|
| Tm1 (Celsius) | 65.5 | 63.6 | 63.6 |
| Tm2 (Celsius) | — | 74.2 | 75.2 |

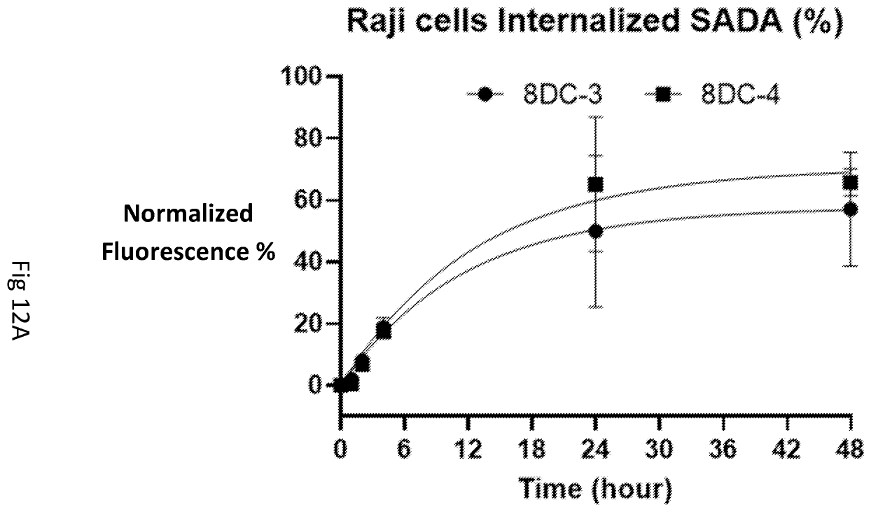

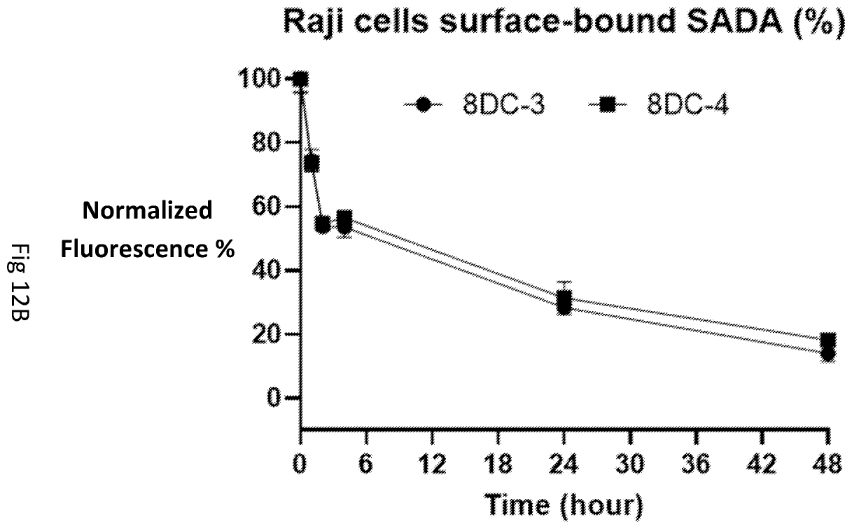

Internalization assay: To assess the internalization of 8DC3 and 8DC4, an internalization assay was performed. First, the antibody was labelled with Alexa Fluor 488 per manufacturer's instructions. Then, CD38(+) Raji cells were stained by the antibody for 30 minutes followed by a wash step. The cells were incubated at 4° C. or 37° C. for up to 48 hours. At pre-determined timepoints, the cells were stained with either a secondary antibody or an Alexa Fluor 488 quencher. The surface-bound and internalized antibody was measured by a flow cytometry. As shown in FIG. 12A-12B, more than 50% of 8DC3 and 8DC4 get internalized after 24 hours followed by a slowdown and plateauing in the rate of internalization afterwards.

### REFERENCES

1. Lee, H. C., Structure and enzymatic functions of human CD38. Mol Med, 2006. 12(11-12): p. 317-23.
2. van de Donk, N., P. G. Richardson, and F. Malavasi, CD38 antibodies in multiple myeloma: back to the future. Blood, 2018. 131(1): p. 13-29.
3. Morandi, F., et al., CD38: A Target for Immunotherapeutic Approaches in Multiple Myeloma. Front Immunol, 2018. 9: p. 2722.
4. Lin, P., et al., Flow cytometric immunophenotypic analysis of 306 cases of multiple myeloma. Am J Clin Pathol, 2004. 121(4): p. 482-8.
5. Chiaretti, S. and R. Foa, T-cell acute lymphoblastic leukemia. Haematologica, 2009. 94(2): p. 160-2.
6. Raetz, E. A. and D. T. Teachey, T-cell acute lymphoblastic leukemia. Hematology Am Soc Hematol Educ Program, 2016. 2016(1): p. 580-588.
7. Naik, J., et al., CD38 as a therapeutic target for adult acute myeloid leukemia and T-cell acute lymphoblastic leukemia. Haematologica, 2019. 104(3): p. e100-e103.
8. Feng, X., et al., Targeting CD38 Suppresses Induction and Function of T Regulatory Cells to Mitigate Immunosuppression in Multiple Myeloma. Clin Cancer Res, 2017. 23(15): p. 4290-4300.
9. Esteve-Sole, A., et al., Characterization of the Highly Prevalent Regulatory CD24(hi)CD38(hi) B-Cell Population in Human Cord Blood. Front Immunol, 2017. 8: p. 201.
10. Chen, L., et al., CD38-Mediated Immunosuppression as a Mechanism of Tumor Cell Escape from PD-1/PD-L1 Blockade. Cancer Discov, 2018. 8(9): p. 1156-1175.
11. Larson, S. M., et al., Radioimmunotherapy of human tumours. Nat Rev Cancer, 2015. 15(6): p. 347-60.
12. Ghose, J., et al., Daratumumab induces CD38 internalization and impairs myeloma cell adhesion. Oncoimmunology, 2018. 7(10): p. e1486948.
13. Raab, M. S., et al., MOR202 alone and in combination with pomalidomide or lenalidomide in relapsed or refractory multiple myeloma: data from clinically relevant cohorts from a phase I/IIa study. J Clin Oncol, 2016. 34(Suppl): p. abstract8012.
14. Ellis, J. H., et al., Engineered anti-CD38 monoclonal antibodies for immunotherapy of multiple myeloma. J Immunol, 1995. 155(2): p. 925-37.
15. Zuch de Zafra, C L er al., Targeting Multiple Myelloma with AMG 424, a Novel Anti-CD38/CD3 Bispecific T Cell-recruiting Antibody Optimized for Cytotoxicity and Cytokine release. Clinical Cancer Research, July 2019, vol. 25, No. 13 p. 3921-3933, doi: 10.1158/1078-0432.CCR-18-2752
16. U.S. Food and Drug Administration. [label] Darzalex https://www.accessdata.fda.gov/drugsatfda_docs/label/2019/761036s020lbl.pdf Accessed 4june 2020
17. ThermoFisher Scientific. [product info] AT13/5 https://www.thermofisher.com/antibody/product/CD38-Antibody-clone-AT13-5-Monoclonal/MA5-16577 Accessed 4june 2020
18. Glenmark Pharma. [Product info] http://www.glenmarkpharma.com/content/gbr-1342 Accessed 4june 2020
19. U.S. Food and Drug Administration [package insert], Yescarta https://www.fda.gov/media/108377/download Accessed 4june 2020
20. U.S. Food and Drug Administration [package insert], Kymriah https://www.fda.gov/files/vaccines %2C %20blood %20%26%20biologics/published/Pa ckage-Insert---KYMRIAH .pdf Accessed 4june 2020
21. U.S. Food and Drug Administration [label], Blincyto https://www.accessdata.fda.gov/drugsatfda_docs/label/2017/125557s008lbl.pdf Accessed 4june 2020

chimeric VL
SEQ ID No: 1
DIQLTQSPSSFSVSLGDRVTITCKASEDIYNRLTWYQQKPGNAPRLLISGATSLETGVPSRFSGSGSGKDYTL
SITSLQTEDVATYYCQQYWSNPYTFGGGTKLEIR
Hu anti-CD38 VL1
SEQ ID No: 2
DIQMTQSPSSLSASVGDRVTITCRASEDIYNRLTWYQQKPGKAPKLLLSGATSLETGVPSRFSGSGSGKDY
TLTISSLOPEDFATYYCQQYWSNPYTFGGGTKVEIK
Hu anti-CD38 VL2
SEQ ID No: 3
DIQMTQSPSSLSASVGDRVTITCRASEDIYNRLAWYQQKPGKAPKLLLSGATSLETGVPSRFSGSGSGTDY
TLTISSLOPEDFATYYCQQYWSNPYTFGGGTKVEIK
Hu anti-CD38 VL3
SEQ ID No: 4
DIQMTQSPSSLSASVGDRVTITCRASEDIYNRLTWYQQKPGKAPKLLLSGATSLETGVPSRFSGSGSGTDY
TLTISSLOPEDFATYYCQQYWSNPYTFGGGTKVEIK
Hu anti-CD38 VL4
SEQ ID No: 5
DIQMTQSPSSLSASVGDRVTITCRASEDIYNRLTWYQQKPGKAPKLLLYGATSLETGVPSRFSGSGSGKDY
TLTISSLOPEDFATYYCQQYWSNPYTFGGGTKVEIK
Hu anti-CD38 VL5
SEQ ID No: 6
DIQLTQSPSSLSASVGDRVTITCKASEDIYNRLTWYQQKPGKAPKLLISGATSLETGVPSRFSGSGSGKDYTF
TISSLOPEDFATYYCQQYWSNPYTFGQGTKLEIK
Daratumumab VL
SEQ ID No: 7
EIVLTQSPATLSLSPGERATLSCRASQSVSSYLAWYQQKPGQAPRLLIYDASNRATGIPARFSGSGSGTDFT
LTISSLEPEDFAVYYCQQRSNWPPTFGQGTKVEIK
chimeric VH
SEQ ID No: 8
QVQLKQSGPGLVHPSQSLSITCTVSGFSLTSYGVHWVRQSPGKGLEWLGVMWRGGSTDYNAAFMSRL
NITKDNSKRQVFFKMNSLQADDTAIYYCAKSMITTGFVMDSWGQGTSVTVSS
Hu anti-CD38 VH1
SEQ ID No: 9
QVQLQESGPGLVKPSETLSLTCTVSGFSLTSYGVHWIRQPPGKGLEWIGVMWRGGSTDYNPSLKSRVTIS
KDNSKRQFSLKLSSVTAADTAVYYCAKSMITTGFVMDSWGQGTLVTVSS
Hu anti-CD38 VH2
SEQ ID No: 10
QVQLQESGPGLVKPSETLSLTCTVSGFSLTSYGVHWVRQPPGKGLEWIGVMWRGGSTDYNPSLKSRVTI
SKDNSKRQFSLKMSSVTAADTAVYYCAKSMITTGFVMDSWGQGTLVTVSS
Hu anti-CD38 VH3
SEQ ID No: 11
QVQLQESGPGLVKPSETLSITCTVSGFSLTSYGVHWIRQPPGKGLEWIGVMWRGGSTDYNPSLKSRVTIS
KDTSKNQFSLKLSSLTAADTAVYYCAKSMITTGFVMDSWGQGTLVTVSS
Hu anti-CD38 VH4
SEQ ID No: 12
QVQLQESGPGLVKPSETLSLTCTVSGFSLTSYGVHWIRQPPGKGLEWIGVMWRGGSTDYNPSLKSRVTIS
VDTSKRQVSLKLSSVTAADTAVYYCAKSMITTGFVMDSWGQGTLVTVSS
Hu anti-CD38 VH5
SEQ ID No: 13
QVQLQESGPGLVKPSETLSLTCTVSGFSLTSYGVHWIRQPPGKGLEWIGVMWRGGSTDYNPSLKSRVTIS
VDTSKNQFSFKLSSVTAADTAIYYCAKSMITTGFVMDSWGQGTLVTVSS
Hu anti-CD38 VH6
SEQ ID No: 14
QVQLQESGPGLVKPSETLSLTCTVSGFSLTSYGVHWIRQPPGKGLEWLGVMWRGGSTNYNPSLKSRVTI
SKDTSKNQFSLKLSSVTAADTAVYYCAKSMITTGFVMDSWGQGTLVTVSS
Hu anti-CD38 VH7
SEQ ID No: 15
QVQLKQSGPGLVKPSETLSLTCTVSGFSLTSYGVHWIRQPPGKGLEWIGVMWRGGSTNYNPSLKSRLTIS
VDTSKNQFSLKLSSVTAADTAVYYCAKSMITTGFVMDSWGQGTLVTVSS
Hu anti-CD38 VH8
SEQ ID No: 16
QVQLQESGPGLVKPSETLSLTCTVSGFSLTSYGVHWVRQPPGKGLEWIGVMWRGGSTDYNAAFKSRVTI
SKDNSKNQVSLKLSSVTAADTAVYYCAKSMITTGFVMDSWGQGTLVTVSS
Daratumumab VH
SEQ ID No: 17
EVQLLESGGGLVQPGGSLRLSCAVSGFTFNSFAMSWVRQAPGKGLEWVSAISGSGGGTYYADSVKGRFT
ISRDNSKNTLYLQMNSLRAEDTAVYFCAKDKILWFGEPVFDYWGQGTLVTVSS
IGKV1-NL1*01:
SEQ ID No: 18
DIQMTQSPSSLSASVGDRVTITCRASQGISNSLAWYQQKPGKAPKLLLYAASRLESGVPSRFSGSGSGTDY
TLTISSLOPEDFATYYCQQYYSTP
IGKJ4*01
SEQ ID No: 19
LTFGGGTKVEIK
8DC1OK Light chain
Anti CD38 VL-CL-(G4S)4-huOKT3 VH-(G4S)6-huOKT3 VL)
SEQ ID No: 20
DIQLTQSPSSFSVSLGDRVTITCKASEDIYNRLTWYQQKPGNAPRLLISGATSLETGVPSRFSGSGSGKDYTL
SITSLQTEDVATYYCQQYWSNPYTFGGGTKLEIRRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSFNRGECG
GGGSGGGGSGGGGSGGGGSQVQLVQSGGGVVQPGRSLRLSCKASGYTFTRYTMHWVRQAPGKCLE
WIGYINPSRGYTNYNQKFKDRFTISRDNSKNTAFLQMDSLRPEDTGVYFCARYYDDHYSLDYWGQGTPV
TVSSGGGGSGGGGSGGGGSGGGGSGGGGSGGGGSDIQMTQSPSSLSASVGDRVTITCSASSSVSYMN
WYQQTPGKAPKRWIYDTSKLASGVPSRFSGSGSGTDYTFTISSLQPEDIATYYCQQWSSNPFTFGCGTKL
QITR
8DC1OK Heavy chain
Anti CD38 VH- CH1-3
SEQ ID No: 21
QVQLKQSGPGLVHPSQSLSITCTVSGFSLTSYGVHWVRQSPGKGLEWLGVMWRGGSTDYNAAFMSRL
NITKDNSKRQVFFKMNSLQADDTAIYYCAKSMITTGFVMDSWGQGTSVTVSSASTKGPSVFPLAPSSKST
SGGTAALGCLVKDYFPEPVTVSWNSGALTSGVHTFPAVLQSSGLYSLSSVVTVPSSSLGTQTYICNVNHKP
SNTKVDKRVEPKSCDKTHTCPPCPAPELLGGPSVFLFPPKPKDTLMISRTPEVTCVVVDVSHEDPEVKFN
WYVDGVEVHNAKTKPREEQYASTYRVVSVLTVLHQDWLNGKEYKCAVSNKALPAPIEKTISKAKGQPRE
PQVYTLPPSRDELTKNQVSLTCLVKGFYPSDIAVEWESNGQPENNYKTTPPVLDSDGSFFLYSKLTVDKSR
WQQGNVFSCSVMHEALHNHYTQKSLSLSPGK
8DC3OK Light chain
Anti CD38 VL6-CL-(G4S)4-huOKT3 VH9-(G4S)6-huOKT3 VL)
SEQ ID No: 22
DIQLTQSPSSLSASVGDRVTITCKASEDIYNRLTWYQQKPGKAPKLLISGATSLETGVPSRFSGSGSGKDYTF
TISSLQPEDFATYYCQQYWSNPYTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSFNRGECG
GGGSGGGGSGGGGSGGGGSQVQLVQSGGGVVQPGRSLRLSCKASGYTFTRYTMHWVRQAPGKCLE
WIGYINPSRGYTNYNQKFKDRFTISRDNSKNTAFLQMDSLRPEDTGVYFCARYYDDHYSLDYWGQGTPV
TVSSGGGGSGGGGSGGGGSGGGGSGGGGSGGGGSDIQMTQSPSSLSASVGDRVTITCSASSSVSYMN
WYQQTPGKAPKRWIYDTSKLASGVPSRFSGSGSGTDYTFTISSLQPEDIATYYCQQWSSNPFTFGCGTKL
QITR
8DC3OK Heavy chain
Anti CD38 VH- CH1-3
SEQ ID No: 23
QVQLQESGPGLVKPSETLSLTCTVSGFSLTSYGVHWVRQPPGKGLEWIGVMWRGGSTDYNAAFKSRVTI
SKDNSKNQVSLKLSSVTAADTAVYYCAKSMITTGFVMDSWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWYV
DGVEVHNAKTKPREEQYASTYRVVSVLTVLHQDWLNGKEYKCAVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGK
DRTOK Light chain
Daratumumab VL-CL-(G4S)4-huOKT3 VH-(G4S)6-huOKT3 VL)
SEQ ID No: 24
EIVLTQSPATLSLSPGERATLSCRASQSVSSYLAWYQQKPGQAPRLLIYDASNRATGIPARFSGSGSGTDFT
LTISSLEPEDFAVYYCQQRSNWPPTFGQGTKVEIKRTVAAPSVFIFPPSDEQLKSGTASVVCLLNNFYPREA
KVQWKVDNALQSGNSQESVTEQDSKDSTYSLSSTLTLSKADYEKHKVYACEVTHQGLSSPVTKSFNRGEC
GGGGSGGGGSGGGGSGGGGSQVQLVQSGGGVVQPGRSLRLSCKASGYTFTRYTMHWVRQAPGKCLE
WIGYINPSRGYTNYNQKFKDRFTISRDNSKNTAFLQMDSLRPEDTGVYFCARYYDDHYSLDYWGQGTPV
TVSSGGGGSGGGGSGGGGSGGGGSGGGGSGGGGSDIQMTQSPSSLSASVGDRVTITCSASSSVSYMN
WYQQTPGKAPKRWIYDTSKLASGVPSRFSGSGSGTDYTFTISSLQPEDIATYYCQQWSSNPFTFGCGTKL
QITR
DRTOK Heavy chain
Daratumumab VH- CH1-3
SEQ ID No: 25
EVQLLESGGGLVQPGGSLRLSCAVSGFTFNSFAMSWVRQAPGKGLEWVSAISGSGGGTYYADSVKGRFT
ISRDNSKNTLYLQMNSLRAEDTAVYFCAKDKILWFGEPVFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKP
SNTKVDKRVEPKSCDKTHTCPPCPAPELLGGPSVFLFPPKPKDTLMISRTPEVTCVVVDVSHEDPEVKFN
WYVDGVEVHNAKTKPREEQYASTYRVVSVLTVLHQDWLNGKEYKCAVSNKALPAPIEKTISKAKGQPRE
PQVYTLPPSRDELTKNQVSLTCLVKGFYPSDIAVEWESNGQPENNYKTTPPVLDSDGSFFLYSKLTVDKSR
WQQGNVFSCSVMHEALHNHYTQKSLSLSPGK
8DC-1
Anti-CD38 scFv-(G4S)4-huC825 anti DOTA scFv-linker-P53-linker-His tag
SEQ ID No: 26
QVQLKQSGPGLVHPSQSLSITCTVSGFSLTSYGVHWVRQSPGKGLEWLGVMWRGGSTDYNAAFMSRL
NITKDNSKRQVFFKMNSLQADDTAIYYCAKSMITTGFVMDSWGQGTSVTVSSGGGGSGGGGSGGGGS
GGGGSGGGGSGGGGSDIQLTQSPSSFSVSLGDRVTITCKASEDIYNRLTWYQQKPGNAPRLLISGATSLET
GVPSRFSGSGSGKDYTLSITSLQTEDVATYYCQQYWSNPYTFGGGTKLEIRGGGGSGGGGSGGGGSGGG
GSHVQLVESGGGLVQPGGSLRLSCAASGFSLTDYGVHWVRQAPGKGLEWLGVIWSGGGTAYNTALISR
FTISRDNSKNTLYLQMNSLRAEDTAVYYCARRGSYPYNYFDAWGCGTLVTVSSGGGGSGGGGSGGGGS
GGGGSGGGGSGGGGSQAVVTQEPSLTVSPGGTVTLTCGSSTGAVTASNYANWVQQKPGQCPRGLIGG
HNNRPPGVPARFSGSLLGGKAALTLLGAQPEDEAEYYCALWYSDHWVIGGGTKLTVLGTPLGDTTHTSG
KPLDGEYFTLQIRGRERFEMFRELNEALELKDAQAGKEPGGSGGAPHHHHHH
8DC-2
Anti-CD38 scFv-(G4S)4-huC825 anti DOTA scFv-linker-P53-linker-His tag
SEQ ID No: 27
QVQLQESGPGLVRPSQTLSLTCTVSGFTFTSYGVHWVRQPPGRGLEWIGVMWRGGSTDYNAAFMSRV
TMLVDTSKNQFSLRLSSVTAADTAVYYCAKSMITTGFVMDSWGQGSLVTVSSGGGGSGGGGSGGGGS
GGGGSGGGGSGGGGSDIQMTQSPSSLSASVGDRVTITCKASEDIYNRLTWYQQKPGKAPKLLISGATSLE
TGVPSRFSGSGSGTDFTFTISSLQPEDIATYYCQQYWSNPYTFGQGTKVEIKGGGGSGGGGSGGGGSGG
GGSHVQLVESGGGLVQPGGSLRLSCAASGFSLTDYGVHWVRQAPGKGLEWLGVIWSGGGTAYNTALIS
RFTISRDNSKNTLYLQMNSLRAEDTAVYYCARRGSYPYNYFDAWGCGTLVTVSSGGGGSGGGGSGGGG
SGGGGSGGGGSGGGGSQAVVTQEPSLTVSPGGTVTLTCGSSTGAVTASNYANWVQQKPGQCPRGLIG
GHNNRPPGVPARFSGSLLGGKAALTLLGAQPEDEAEYYCALWYSDHWVIGGGTKLTVLGTPLGDTTHTS
GKPLDGEYFTLQIRGRERFEMFRELNEALELKDAQAGKEPGGSGGAPHHHHHH
8DC-3
Anti-CD38 scFv-(G4S)4-huC825 anti DOTA scFv-linker-P53-linker-His tag
SEQ ID No: 28
QVQLQESGPGLVKPSETLSLTCTVSGFSLTSYGVHWVRQPPGKGLEWIGVMWRGGSTDYNAAFKSRVTI
SKDNSKNQVSLKLSSVTAADTAVYYCAKSMITTGFVMDSWGQGTLVTVSSGGGGSGGGGSGGGGSGG
GGSGGGGSGGGGSDIQLTQSPSSLSASVGDRVTITCKASEDIYNRLTWYQQKPGKAPKLLISGATSLETGV
PSRFSGSGSGKDYTFTISSLQPEDFATYYCQQYWSNPYTFGQGTKLEIKGGGGSGGGGSGGGGSGGGGS
HVQLVESGGGLVQPGGSLRLSCAASGFSLTDYGVHWVRQAPGKGLEWLGVIWSGGGTAYNTALISRFTI
SRDNSKNTLYLQMNSLRAEDTAVYYCARRGSYPYNYFDAWGCGTLVTVSSGGGGSGGGGSGGGGSGG
GGSGGGGSGGGGSQAVVTQEPSLTVSPGGTVTLTCGSSTGAVTASNYANWVQQKPGQCPRGLIGGHN
NRPPGVPARFSGSLLGGKAALTLLGAQPEDEAEYYCALWYSDHWVIGGGTKLTVLGTPLGDTTHTSGKPL
DGEYFTLQIRGRERFEMFRELNEALELKDAQAGKEPGGSGGAPHHHHHH
8DC-4
Anti-CD38 scFv-(G4S)4-huC825 anti DOTA scFv-linker-P53-linker-His tag
SEQ ID No: 29
QVQLQESGPGLVKPSETLSLTCTVSGFSLTSYGVHWVRQPPGKCLEWIGVMWRGGSTDYNAAFKSRVTI
SKDNSKNQVSLKLSSVTAADTAVYYCAKSMITTGFVMDSWGQGTLVTVSSGGGGSGGGGSGGGGSGG
GGSGGGGSGGGGSDIQLTQSPSSLSASVGDRVTITCKASEDIYNRLTWYQQKPGKAPKLLISGATSLETGV
PSRFSGSGSGKDYTFTISSLQPEDFATYYCQQYWSNPYTFGCGTKLEIKGGGGSGGGGSGGGGSGGGGS
HVQLVESGGGLVQPGGSLRLSCAASGFSLTDYGVHWVRQAPGKGLEWLGVIWSGGGTAYNTALISRFTI
SRDNSKNTLYLQMNSLRAEDTAVYYCARRGSYPYNYFDAWGCGTLVTVSSGGGGSGGGGSGGGGSGG
GGSGGGGSGGGGSQAVVTQEPSLTVSPGGTVTLTCGSSTGAVTASNYANWVQQKPGQCPRGLIGGHN
NRPPGVPARFSGSLLGGKAALTLLGAQPEDEAEYYCALWYSDHWVIGGGTKLTVLGTPLGDTTHTSGKPL
DGEYFTLQIRG RERFEMFRELNEALELKDAQAGKEPGGSGGAPHHHHHH
8DC-5
Anti-CD38 scFv-(G4S)4-huC825 anti DOTA scFv-linker-P53-linker-His tag
SEQ ID No: 30
DIQLTQSPSSLSASVGDRVTITCKASEDIYNRLTWYQQKPGKAPKLLISGATSLETGVPSRFSGSGSGKDYTF
TISSLQPEDFATYYCQQYWSNPYTFGCGTKLEIKGGGGSGGGGSGGGGSGGGGSGGGGSGGGGSQVQ
LQESGPGLVKPSETLSLTCTVSGFSLTSYGVHWVRQPPGKCLEWIGVMWRGGSTDYNAAFKSRVTISKD
NSKNQVSLKLSSVTAADTAVYYCAKSMITTGFVMDSWGQGTLVTVSSGGGGSGGGGSGGGGSGGGGS
HVQLVESGGGLVQPGGSLRLSCAASGFSLTDYGVHWVRQAPGKGLEWLGVIWSGGGTAYNTALISRFTI
SRDNSKNTLYLQMNSLRAEDTAVYYCARRGSYPYNYFDAWGCGTLVTVSSGGGGSGGGGSGGGGSGG
GGSGGGGSGGGGSQAVVTQEPSLTVSPGGTVTLTCGSSTGAVTASNYANWVQQKPGQCPRGLIGGHN
NRPPGVPARFSGSLLGGKAALTLLGAQPEDEAEYYCALWYSDHWVIGGGTKLTVLGTPLGDTTHTSGKPL
DGEYFTLQIRGRERFEMFRELNEALELKDAQAGKEPGGSGGAPHHHHHH
AT13.5 CDRL1 IMGT
SEQ ID No: 31
EDIYNR
AT13.5 CDRL2 IMGT
SEQ ID No: 32
GA
AT13.5 CDRL3 IMGT
SEQ ID No: 33
QQYWSNPYT
AT13.5 CDRH1 IMGT
SEQ ID No: 34
GFSLTSYG
AT13.5 CDRH2 IMGT
SEQ ID No: 35
MWRGGST
AT13.5 CDRH3 IMGT
SEQ ID No: 36
AKSMITTGFVMDS
Hu anti-CD38 VL7
SEQ ID No: 37
DIQMTQSPSSLSASVGDRVTITCKASEDIYNRLTWYQQKPGKAPKLLISGATSLETGVPSRFSGSGSGTDFT
FTISSLQPEDIATYYCQQYWSNPYTFGQGTKVEIK
Hu anti-CD38 VL8
SEQ ID No: 38
DIQLTQSPSSLSASVGDRVTITCKASEDIYNRLTWYQQKPGKAPKLLISGATSLETGVPSRFSGSGSGKDYTF
TISSLOPEDFATYYCQQYWSNPYTFGCGTKLEIK
Hu anti-CD38 VH10
SEQ ID No: 39
QVQLQESGPGLVRPSQTLSLTCTVSGFTFTSYGVHWVRQPPGRGLEWIGVMWRGGSTDYNAAFMSRV
TMLVDTSKNQFSLRLSSVTAADTAVYYCAKSMITTGFVMDSWGQGSLVTVSS
Hu anti-CD38 VH11
SEQ ID No: 40
QVQLQESGPGLVKPSETLSLTCTVSGFSLTSYGVHWVRQPPGKCLEWIGVMWRGGSTDYNAAFKSRVTI
SKDNSKNQVSLKLSSVTAADTAVYYCAKSMITTGFVMDSWGQGTLVTVSS
IGKV1-NL1*01
SEQ ID No: 41
ccagtgctga gttactgaga tgaaccagcc cttcagctgt gcccagcatg ccctgccccc tgctcatttg catgttccta
cagcacatcc tettgccctg aacacttatt aataggctgg ccacactccg tgcatgagtc agaccctgtc aggacacagc
atggacatga gggtccccgc tcagctcctg gggctcctgc tgctctggct cccaggtaag gatggagaac actaggaatt
tactcagcca gtgtgctcag tactgtctgc ctatttaggg aagttacctt actacatgat taattgtgta gacatttgtt
tttatgtttc caatctcagg taccagatgt gacatccaga tgacccagtc tccatcctcc ctgtctgcat ctgtaggaga
cagagtcacc atcacttgcc gggcgagtca gggcattagc aattctttag cctggtatca gcagaaacca gggaaagccc
ctaagctcct getctatgct gcatccagat tggaaagtgg ggtcccatcc aggttcagtg gcagtggatc tgggacggat
tacactctca ccatcagcag cctgcagcct gaagattttg caacttatta ctgtcaacag tattatagta cccctcccac
agtgttaca
IGKJ4*01
SEQ ID No: 42
gatccctcac tgtggctcac tttcggcgga gggaccaagg tggagatcaa acgtaagtgc actttcctaa tgctttttct
tataaggttt taaatttgga gogtttttgt gtttgagata ttagctcagg tcaattccaa agagtaccag attctttcaa
aaagtcagat gagtaaggga tagaaaatta gttcatctta aggaacagcc aagcgctagc cagttaagtg aggcatctca
attgcaagat tttctctgca toggtcaggt tagtgatatt aacagcgaaa agagattttt
IGHV4-4*08
SEQ ID No: 43
gggcatggct agttgaggcc ccaggaagag aactgagttc tcaaagggca aagcaagcat cctcatccca gggcgagcct
aaaagactgg ggcctccctc atcccttttc acctctttat acaaaggcac cacctacatg caaatcctca cttaggcacc
cataggaaac caccacacat ttccttaaat tcagggtcca gctcacatgg gaaatacttt ctgagagtcc tggacctcct
gcacaagaac atgaaacacc tgtggttctt cctcctcctg gtggcagctc ccagatgtga gtgtctcaag gctgcagaca
tggggatatg ggaggtgcct ctgatcccag ggctcactgt gggtctctct gttcacaggg gtcctgtccc aggtgcagct
gcaggagtcg ggcccaggac tggtgaagcc ttoggagacc ctgtccctca cctgcactgt ctctggtggc tccatcagta
gttactactg gagctggatc cggcagcccc cagggaaggg actggagtgg attgggtata tctataccag tgggagcacc
aactacaacc cctccctcaa gagtcgagtc accatatccg tagacacgtc caagaaccag ttctccctga agctgagctc
tgtgaccgcc gcagacacgg ccgtgtatta ctgtgcgaga gacacagtga ggggaggtga gtgtgagccc agacacaaac
ctccctgcag ggaggcggag gggaccggcg caggtgctgc tcagcgccag cagggggcgc gcggggccca cagagcaaga
ggccgggtct ggagcaggtg cagggagggc ggggcttcct catcagctca atgctctccc tcctcgccag gacctcagct
gtccccaggg ctcctctttc tttattatct gtggttctgc ttcctcacat
IGHJ5*01
SEQ ID No: 44
gacccatttcgagcgtcctgcacgggcacaggtttgtgtctgggtctaggaacggactgtgtccctgtgtgatgcttttgatgtctggg
gccaagggacaatggtcaccgtctcttcaggtaagatgggctttccttctgcctcctttctctggccccagcgtcctctgtcctggagct
gggagataatgtccgggggcctccttggtctgcgctgggccatgtggggcctccggggctccttctccggctgtttgggaccacgttca
gcagaaggcctttctttgggaactgggactctgctgctggggcaaagggtgggcagagtcatgcttgtgctggggacaaaatgacct
tgggacacggggcttggctgccacggccggcccgggacagtcggagagtcaggtttttgtgcaccccttaatggggcctcccacaat
gtggctactttgactactggggccaagggaccctggtcaccgtctcctcaggtgagtcctcacaacctctctcctcctttaactctgaa
gggttttgttgacttttgggggaataagggtgctgggggcctgccaagagagccccggagcagccctgggggctgcaggaggcctg
aggcaacagcggcacacacagacgaggggcaagggtctccagatgctccttcctcctgagccagcagcacgggttcgtctcggcgc
cagggccaccctaggcggaggttcgtgtcttctctgagccaggagcacgggttctctcgcaggcaccctgtgcctctggggtccaatg
cccaacaaccccggccctccccgggctcagtctgagagggtcccagggacgtgcggggcgccggttctttgtcggggtctggcattgt
tgtcacaatgtgacaactggttcgactcctggggccaaggaaccctggtcaccgtctcctcaggtgagtcctcaccaccccctctctga
gtccacttagggagactcagcttgcagggtct
HuC825 anti DOTA scFv
SEQ ID No. 45
HVQLVESGGGLVQPGGSLRLSCAASGFSLTDYGVHWVRQAPGKGLEWLGVIWSGGGTAYNTALISRFTI
SRDNSKNTLYLQMNSLRAEDTAVYYCARRGSYPYNYFDAWGCGTLVTVSSGGGGSGGGGSGGGGSGG
GGSGGGGSGGGGSQAVVTQEPSLTVSPGGTVTLTCGSSTGAVTASNYANWVQQKPGQCPRGLIGGHN
NRPPGVPARFSGSLLGGKAALTLLGAQPEDEAEYYCALWYSDHWVIGGGTKLTVLG
P53
SEQ ID No: 46
KPLDGEYFTLQIRGRERFEMFRELNEALELKDAQAGKEP
(G4S)4 linker
SEQ ID No: 47
GGGGSGGGGSGGGGSGGGGS
Linker
SEQ ID No: 48
TPLGDTTHT
Linker
SEQ ID No: 49
GGSGGAP
His tag
SEQ ID No: 50
HHHHHH
IGHV4-4*08
SEQ ID No: 51
QVQLQESGPGLVKPSETLSLTCTVSGGSISSYYWSWIRQPPGKGLEWIGYIYTSGSTNYNPSLKSRVTISVD
TSKNQFSLKLSSVTAADTAVYYCAR
IGHJ5*01
SEQ ID No: 52
NWFDSWGQGTLVTVSS
HuOKT3 VH
SEQ ID No: 53
QVQLVQSGGGVVQPGRSLRLSCKASGYTFTRYTMHWVRQAPGKCLEWIGYINPSRGYTNYNQKFKDRF
TISRDNSKNTAFLQMDSLRPEDTGVYFCARYYDDHYSLDYWGQGTPVTVSS
HuOKT3 VL
SEQ ID No: 54
DIQMTQSPSSLSASVGDRVTITCSASSSVSYMNWYQQTPGKAPKRWIYDTSKLASGVPSRFSGSGSGTDY
TFTISSLQPEDIATYYCQQWSSNPFTFGCGTKLQITR
Linker
SEQ ID No: 55
GGGGSGGGGSGGGGSGGGGSGGGGSGGGGS
CD38 Isoform 1
SEQ ID No. 56
MANCEFSPVSGDKPCCRLSRRAQLCLGVSILVLILVVVLAVVVPRWRQQWSGPGTTKRFPETVLARCVKY
TEIHPEMRHVDCQSVWDAFKGAFISKHPCNITEEDYQPLMKLGTQTVPCNKILLWSRIKDLAHQFT
RDMFTLEDTLLGYLADDLTWCGEFNTSKINYQSCPDWRKDCSNNPVSVFWKTVSRRFAEAACDVVHV
MLNGSRSKIFDKNSTFGSVEVHNLQPEKVQTLEAWVIHGGREDSRDLCQDPTIKELESIISKRNIQFSCKNI
YRPDKFLQCVKNPEDSSCTSEI
CD38 Isoform 2
SEQ ID No. 57
MANCEFSPVSGDKPCCRLSRRAQLCLGVSILVLILVVVLAVVVPRWRQQWSGPGTTKRFPETVLARCVKY
TEIHPEMRHVDCQSVWDAFKGAFISKHPCNITEEDYQPLMKLGTQTVPCNKK

The content of the ASCII text file of the sequence listing named “Substitute-Sequence-Listing-12397-1702”, having a size of 88.7 kb and a creation date of 19 Jun. 2023, and electronically submitted via EFS-Web on 20 Jun. 2023, is incorporated herein by reference in its entirety.

1.-116. (canceled)

117. An antibody or antigen binding fragment thereof, capable of binding to CD3 8 antigen, wherein said antibody or antigen binding fragment comprises at least one sequence selected from the group consisting of a heavy chain variable region CDR1 according to SEQ ID NO: 34, a heavy chain variable region CDR2 according to SEQ IN NO: 35, a heavy chain variable region CDR3 according to SEQ IN NO: 36, a light chain variable region CDR1 according to SEQ ID NO: 31, a light chain variable region CDR2 according to SEQ ID NO: 32 and a light chain variable region CDR3 according to SEQ ID NO: 33, wherein said antibody comprises sequences of human origin.

118. The antibody or antigen binding fragment thereof according to claim 117, wherein said antibody or antigen binding fragment comprises a heavy chain or variable heavy chain sequence that has at least about 80%, about 85%, about 90%, about 95%, about 96%, about 97%, about 98% or about 99% identity to a sequence of SEQ ID NO: 16.

119. The antibody or antibody fragment according to claim 117, comprising a light chain or variable light chain sequence that has at least about 80%, about 85%, about 90%, about 95%, about 96%, about 97%, about 98% or about 99% sequence identity to the sequence of SEQ ID NO: 6.

120. The antibody or antigen binding fragment thereof according to claim 117, wherein said antibody or antigen binding fragment is radiolabeled with a radioactive isotope.

121. A self-assembly disassembly (SADA) polypeptide, wherein said polypeptide is linked to an antibody or antigen binding fragment according to claim 117.

122. The SADA polypeptide according to claim 121, wherein said antibody is a scFv, and wherein said scFv is linked to a second scFv, and wherein said second scFv is capable of binding to DOTA and/or DTPA.

123. The SADA polypeptide according to claim 122, capable of binding to CD38 antigen, wherein said antibody or antigen binding fragment comprises a sequence that has at least about 80%, about 85%, about 90%, about 95%, about 96%, about 97%, about 98% or about 99% identity to a sequence selected from the sequences set forth in SEQ ID NOS: 28-30.

124. The SADA polypeptide of claim 123 comprising a sequence selected from SEQ ID NOS: 28-30.

125. The SADA polypeptide of claim 123 comprising amino acids 1-592 of a sequence selected from SEQ ID NOS: 28-30.

126. An isolated nucleic acid molecule encoding an antibody or antigen binding fragment according to claim 117 or a SADA polypeptide linked to an antibody or antigen binding fragment according to claim 117.

127. A recombinant vector comprising the isolated nucleic acid molecule of claim 126.

128. A host cell comprising the recombinant vector of claim 127.

129. A pharmaceutical composition comprising an antibody or antigen binding fragment according to claim 117 or a SADA polypeptide linked to an antibody or antigen binding fragment according to claim 117.

130. A method for the production of an antibody or antigen binding fragment thereof according to claim 117, comprising the steps of:
a. culturing a host cell comprising a nucleic acid encoding said antibody or antigen binding fragment thereofError! Reference source not found. in a culture medium under conditions allowing expression of the antibody or fragment thereof; and
b. separating the antibody or fragment thereof from the culture medium.

131. A method of treating, preventing, alleviating and/or diagnosing the symptoms of a medical condition in a subject, comprising administering the SADA polypeptide according to claim 121, wherein said medical condition is characterized by expression of CD38 antigen.

132. The method according to claim 131, wherein said medical condition is a cancer, metastasis, or an autoimmune disease.

133. The method according claim 132, wherein said cancer or metastasis is selected from the group consisting of a multiple myeloma (MM), an AL amyloidosis, an acute myeloid leukemia (AML), a myelodysplastic syndrome (MDS), a proliferative glomerulonephritis with monoclonal immune deposit, a C3 glomerulopathy associated with monoclonal gammopathy, a squamous cell carcinoma, a colon cancer, a non-small cell lung cancer, a mantle cell lymphoma, a diffuse large B-cell lymphoma, a follicular lymphoma, a NK-/T-cell lymphoma, a B-cell non-Hodgkin lymphoma, a T-cell acute lymphoblastic leukemia, a B-cell acute lymphoblastic leukemia, a chronic lymphocytic leukemia (CLL), a Waldenström macroglobulinemia, a solid cancer, a cancer with immunomodulatory activity and a cancer expressing CD38.

134. The method according to claim 132, wherein said autoimmune disease is selected from the group consisting of rheumatoid arthritis, multiple sclerosis, paraneoplastic syndromes, systemic lupus erythematosus, type 2 diabetes, Pemphigus Vulgaris, autoimmune hemolytic anemia, allergy and graft-versus-host disease.


SEQUENCE LISTING (57 sequences)

SEQ ID NO: 1 - protein, 107 aa, Artificial Sequence
  chimeric VL
DIQLTQSPSSFSVSLGDRVTITCKASEDIYNRLTWYQQKPGNAPRLLISGATSLETGVPSRFSGSGSGKDYTLSITSLQTEDVATYYCQQYWSNPYTFGGGTKLEIR

SEQ ID NO: 2 - protein, 107 aa, Artificial Sequence
  Hu anti-CD38 VL1
DIQMTQSPSSLSASVGDRVTITCRASEDIYNRLTWYQQKPGKAPKLLLSGATSLETGVPSRFSGSGSGKDYTLTISSLQPEDFATYYCQQYWSNPYTFGGGTKVEIK

SEQ ID NO: 3 - protein, 107 aa, Artificial Sequence
  Hu anti-CD38 VL2
DIQMTQSPSSLSASVGDRVTITCRASEDIYNRLAWYQQKPGKAPKLLLSGATSLETGVPSRFSGSGSGTDYTLTISSLQPEDFATYYCQQYWSNPYTFGGGTKVEIK

SEQ ID NO: 4 - protein, 107 aa, Artificial Sequence
  Hu anti-CD38 VL3
DIQMTQSPSSLSASVGDRVTITCRASEDIYNRLTWYQQKPGKAPKLLLSGATSLETGVPSRFSGSGSGTDYTLTISSLQPEDFATYYCQQYWSNPYTFGGGTKVEIK

SEQ ID NO: 5 - protein, 107 aa, Artificial Sequence
  Hu anti-CD38 VL4
DIQMTQSPSSLSASVGDRVTITCRASEDIYNRLTWYQQKPGKAPKLLLYGATSLETGVPSRFSGSGSGKDYTLTISSLQPEDFATYYCQQYWSNPYTFGGGTKVEIK

SEQ ID NO: 6 - protein, 107 aa, Artificial Sequence
  Hu anti-CD38 VL5
DIQLTQSPSSLSASVGDRVTITCKASEDIYNRLTWYQQKPGKAPKLLISGATSLETGVPSRFSGSGSGKDYTFTISSLQPEDFATYYCQQYWSNPYTFGQGTKLEIK

SEQ ID NO: 7 - protein, 107 aa, Homo Sapiens [sequence omitted]

SEQ ID NO: 8 - protein, 119 aa, Artificial Sequence
  chimeric VH
QVQLKQSGPGLVHPSQSLSITCTVSGFSLTSYGVHWVRQSPGKGLEWLGVMWRGGSTDYNAAFMSRLNITKDNSKRQVFFKMNSLQADDTAIYYCAKSMITTGFVMDSWGQGTSVTVSS

SEQ ID NO: 9 - protein, 119 aa, Artificial Sequence
  Hu anti-CD38 VH1
QVQLQESGPGLVKPSETLSLTCTVSGFSLTSYGVHWIRQPPGKGLEWIGVMWRGGSTDYNPSLKSRVTISKDNSKRQFSLKLSSVTAADTAVYYCAKSMITTGFVMDSWGQGTLVTVSS

SEQ ID NO: 10 - protein, 119 aa, Artificial Sequence
  Hu anti-CD38 VH2
QVQLQESGPGLVKPSETLSLTCTVSGFSLTSYGVHWVRQPPGKGLEWIGVMWRGGSTDYNPSLKSRVTISKDNSKRQFSLKMSSVTAADTAVYYCAKSMITTGFVMDSWGQGTLVTVSS

SEQ ID NO: 11 - protein, 119 aa, Artificial Sequence
  Hu anti-CD38 VH3
QVQLQESGPGLVKPSETLSITCTVSGFSLTSYGVHWIRQPPGKGLEWIGVMWRGGSTDYNPSLKSRVTISKDTSKNQFSLKLSSLTAADTAVYYCAKSMITTGFVMDSWGQGTLVTVSS

SEQ ID NO: 12 - protein, 119 aa, Artificial Sequence
  Hu anti-CD38 VH4
QVQLQESGPGLVKPSETLSLTCTVSGFSLTSYGVHWIRQPPGKGLEWIGVMWRGGSTDYNPSLKSRVTISVDTSKRQVSLKLSSVTAADTAVYYCAKSMITTGFVMDSWGQGTLVTVSS

SEQ ID NO: 13 - protein, 119 aa, Artificial Sequence
  Hu anti-CD38 VH5
QVQLQESGPGLVKPSETLSLTCTVSGFSLTSYGVHWIRQPPGKGLEWIGVMWRGGSTDYNPSLKSRVTISVDTSKNQFSFKLSSVTAADTAIYYCAKSMITTGFVMDSWGQGTLVTVSS

SEQ ID NO: 14 - protein, 119 aa, Artificial Sequence
  Hu anti-CD38 VH6
QVQLQESGPGLVKPSETLSLTCTVSGFSLTSYGVHWIRQPPGKGLEWLGVMWRGGSTNYNPSLKSRVTISKDTSKNQFSLKLSSVTAADTAVYYCAKSMITTGFVMDSWGQGTLVTVSS

SEQ ID NO: 15 - protein, 119 aa, Artificial Sequence
  Hu anti-CD38 VH7
QVQLKQSGPGLVKPSETLSLTCTVSGFSLTSYGVHWIRQPPGKGLEWIGVMWRGGSTNYNPSLKSRLTISVDTSKNQFSLKLSSVTAADTAVYYCAKSMITTGFVMDSWGQGTLVTVSS

SEQ ID NO: 16 - protein, 119 aa, Artificial Sequence
  Hu anti-CD38 VH8
QVQLQESGPGLVKPSETLSLTCTVSGFSLTSYGVHWVRQPPGKGLEWIGVMWRGGSTDYNAAFKSRVTISKDNSKNQVSLKLSSVTAADTAVYYCAKSMITTGFVMDSWGQGTLVTVSS

SEQ ID NO: 17 - protein, 122 aa, Homo Sapiens [sequence omitted]

SEQ ID NO: 18 - protein, 95 aa, Artificial Sequence
  IGKV1-NL1*01
DIQMTQSPSSLSASVGDRVTITCRASQGISNSLAWYQQKPGKAPKLLLYAASRLESGVPSRFSGSGSGTDYTLTISSLQPEDFATYYCQQYYSTP

SEQ ID NO: 19 - protein, 12 aa, Artificial Sequence
  IGKJ4*01
LTFGGGTKVEIK

SEQ ID NO: 20 - protein, 490 aa, Artificial Sequence
  8DC1OK Light chain
DIQLTQSPSSFSVSLGDRVTITCKASEDIYNRLTWYQQKPGNAPRLLISGATSLETGVPSRFSGSGSGKDYTLSITSLQTEDVATYYCQQYWSNPYTFGGGTKLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 21 - protein, 449 aa, Artificial Sequence
  8DC1OK Heavy chain
QVQLKQSGPGLVHPSQSLSITCTVSGFSLTSYGVHWVRQSPGKGLEWLGVMWRGGSTDYNAAFMSRLNITKDNSKRQVFFKMNSLQADDTAIYYCAKSMITTGFVMDSWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 490 aa, Artificial Sequence
  8DC3OK Light chain
DIQLTQSPSSLSASVGDRVTITCKASEDIYNRLTWYQQKPGKAPKLLISGATSLETGVPSRFSGSGSGKDYTFTISSLQPEDFATYYCQQYWSNPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 23 - protein, 449 aa, Artificial Sequence
  8DC3OK Heavy chain
QVQLQESGPGLVKPSETLSLTCTVSGFSLTSYGVHWVRQPPGKGLEWIGVMWRGGSTDYNAAFKSRVTISKDNSKNQVSLKLSSVTAADTAVYYCAKSMITTGFVMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 489 aa, Artificial Sequence
  DRTOK Light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQIT

SEQ ID NO: 25 - protein, 452 aa, Artificial Sequence
  DRTOK Heavy chain
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 598 aa, Artificial Sequence
  8DC-1
QVQLKQSGPGLVHPSQSLSITCTVSGFSLTSYGVHWVRQSPGKGLEWLGVMWRGGSTDYNAAFMSRLNITKDNSKRQVFFKMNSLQADDTAIYYCAKSMITTGFVMDSWGQGTSVTVSSGGGGSGGGGSGGGGSGGGGSGGGGSGGGGSDIQLTQSPSSFSVSLGDRVTITCKASEDIYNRLTWYQQKPGNAPRLLISGATSLETGVPSRFSGSGSGKDYTLSITSLQTEDVATYYCQQYWSNPYTFGGGTKLEIRGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAPHHHHHH

SEQ ID NO: 27 - protein, 598 aa, Artificial Sequence
  8DC-2
QVQLQESGPGLVRPSQTLSLTCTVSGFTFTSYGVHWVRQPPGRGLEWIGVMWRGGSTDYNAAFMSRVTMLVDTSKNQFSLRLSSVTAADTAVYYCAKSMITTGFVMDSWGQGSLVTVSSGGGGSGGGGSGGGGSGGGGSGGGGSGGGGSDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAPHHHHHH

SEQ ID NO: 28 - protein, 598 aa, Artificial Sequence
  8DC-3
QVQLQESGPGLVKPSETLSLTCTVSGFSLTSYGVHWVRQPPGKGLEWIGVMWRGGSTDYNAAFKSRVTISKDNSKNQVSLKLSSVTAADTAVYYCAKSMITTGFVMDSWGQGTLVTVSSGGGGSGGGGSGGGGSGGGGSGGGGSGGGGSDIQLTQSPSSLSASVGDRVTITCKASEDIYNRLTWYQQKPGKAPKLLISGATSLETGVPSRFSGSGSGKDYTFTISSLQPEDFATYYCQQYWSNPYTFGQGTKLEIKGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAPHHHHHH

SEQ ID NO: 29 - protein, 598 aa, Artificial Sequence
  8DC-4
QVQLQESGPGLVKPSETLSLTCTVSGFSLTSYGVHWVRQPPGKCLEWIGVMWRGGSTDYNAAFKSRVTISKDNSKNQVSLKLSSVTAADTAVYYCAKSMITTGFVMDSWGQGTLVTVSSGGGGSGGGGSGGGGSGGGGSGGGGSGGGGSDIQLTQSPSSLSASVGDRVTITCKASEDIYNRLTWYQQKPGKAPKLLISGATSLETGVPSRFSGSGSGKDYTFTISSLQPEDFATYYCQQYWSNPYTFGCGTKLEIKGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAPHHHHHH

SEQ ID NO: 30 - protein, 598 aa, Artificial Sequence
  8DC-5
DIQLTQSPSSLSASVGDRVTITCKASEDIYNRLTWYQQKPGKAPKLLISGATSLETGVPSRFSGSGSGKDYTFTISSLQPEDFATYYCQQYWSNPYTFGCGTKLEIKGGGGSGGGGSGGGGSGGGGSGGGGSGGGGSQVQLQESGPGLVKPSETLSLTCTVSGFSLTSYGVHWVRQPPGKCLEWIGVMWRGGSTDYNAAFKSRVTISKDNSKNQVSLKLSSVTAADTAVYYCAKSMITTGFVMDSWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAPHHHHHH

SEQ ID NO: 31 - protein, 6 aa, Mus musculus
EDIYNR

SEQ ID NO: 32 - protein, 2 aa, Mus musculus
GA

SEQ ID NO: 33 - protein, 9 aa, Mus musculus
QQYWSNPYT

SEQ ID NO: 34 - protein, 8 aa, Mus musculus
GFSLTSYG

SEQ ID NO: 35 - protein, 7 aa, Mus musculus
MWRGGST

SEQ ID NO: 36 - protein, 13 aa, Mus musculus
AKSMITTGFVMDS

SEQ ID NO: 37 - protein, 107 aa, Artificial Sequence
  Hu anti-CD38 VL7
DIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIK

SEQ ID NO: 38 - protein, 107 aa, Artificial Sequence
  Hu anti-CD38 VL8
DIQLTQSPSSLSASVGDRVTITCKASEDIYNRLTWYQQKPGKAPKLLISGATSLETGVPSRFSGSGSGKDYTFTISSLQPEDFATYYCQQYWSNPYTFGCGTKLEIK

SEQ ID NO: 39 - protein, 119 aa, Artificial Sequence
  Hu anti-CD38 VH10
QVQLQESGPGLVRPSQTLSLTCTVSGFTFTSYGVHWVRQPPGRGLEWIGVMWRGGSTDYNAAFMSRVTMLVDTSKNQFSLRLSSVTAADTAVYYCAKSMITTGFVMDSWGQGSLVTVSS

SEQ ID NO: 40 - protein, 119 aa, Artificial Sequence
  Hu anti-CD38 VH11
QVQLQESGPGLVKPSETLSLTCTVSGFSLTSYGVHWVRQPPGKCLEWIGVMWRGGSTDYNAAFKSRVTISKDNSKNQVSLKLSSVTAADTAVYYCAKSMITTGFVMDSWGQGTLVTVSS

SEQ ID NO: 41 - DNA, 649 bp, Artificial Sequence
  IGKV1-NL1*01
ccagtgctgagttactgagatgaaccagcccttcagctgtgcccagcatgccctgccccctgctcatttgcatgttcctacagcacatcctcttgccctgaacacttattaataggctggccacactccgtgcatgagtcagaccctgtcaggacacagcatggacatgagggtccccgctcagctcctggggctcctgctgctctggctcccaggtaaggatggagaacactaggaatttactcagccagtgtgctcagtactgtctgcctatttagggaagttaccttactacatgattaattgtgtagacatttgtttttatgtttccaatctcaggtaccagatgtgacatccagatgacccagtctccatcctccctgtctgcatctgtaggagacagagtcaccatcacttgccgggcgagtcagggcattagcaattctttagcctggtatcagcagaaaccagggaaagcccctaagctcctgctctatgctgcatccagattggaaagtggggtcccatccaggttcagtggcagtggatctgggacggattacactctcaccatcagcagcctgcagcctgaagattttgcaacttattactgtcaacagtattatagtacccctcccacagtgttaca

SEQ ID NO: 42 - DNA, 300 bp, Artificial Sequence
  IGKJ4*01
gatccctcactgtggctcactttcggcggagggaccaaggtggagatcaaacgtaagtgcactttcctaatgctttttcttataaggttttaaatttggagcgtttttgtgtttgagatattagctcaggtcaattccaaagagtaccagattctttcaaaaagtcagatgagtaagggatagaaaattagttcatcttaaggaacagccaagcgctagccagttaagtgaggcatctcaattgcaagattttctctgcatcggtcaggttagtgatattaacagcgaaaagagattttt

SEQ ID NO: 43 - DNA, 930 bp, Artificial Sequence
  IGHV4-4*08
gggcatggctagttgaggccccaggaagagaactgagttctcaaagggcaaagcaagcatcctcatcccagggcgagcctaaaagactggggcctccctcatcccttttcacctctttatacaaaggcaccacctacatgcaaatcctcacttaggcacccataggaaaccaccacacatttccttaaattcagggtccagctcacatgggaaatactttctgagagtcctggacctcctgcacaagaacatgaaacacctgtggttcttcctcctcctggtggcagctcccagatgtgagtgtctcaaggctgcagacatggggatatgggaggtgcctctgatcccagggctcactgtgggtctctctgttcacaggggtcctgtcccaggtgcagctgcaggagtcgggcccaggactggtgaagccttcggagaccctgtccctcacctgcactgtctctggtggctccatcagtagttactactggagctggatccggcagcccccagggaagggactggagtggattgggtatatctataccagtgggagcaccaactacaacccctccctcaagagtcgagtcaccatatccgtagacacgtccaagaaccagttctccctgaagctgagctctgtgaccgccgcagacacggccgtgtattactgtgcgagagacacagtgaggggaggtgagtgtgagcccagacacaaacctccctgcagggaggcggaggggaccggcgcaggtgctgctcagcgccagcagggggcgcgcggggcccacagagcaagaggccgggtctggagcaggtgcagggagggcggggcttcctcatcagctcaatgctctccctcctcgccaggacctcagctgtccccagggctcctctttctttattatctgtggttctgcttcctcacat

SEQ ID NO: 44 - DNA, 1000 bp, Artificial Sequence
  IGHJ5*01
gacccatttcgagcgtcctgcacgggcacaggtttgtgtctgggtctaggaacggactgtgtccctgtgtgatgcttttgatgtctggggccaagggacaatggtcaccgtctcttcaggtaagatgggctttccttctgcctcctttctctggccccagcgtcctctgtcctggagctgggagataatgtccgggggcctccttggtctgcgctgggccatgtggggcctccggggctccttctccggctgtttgggaccacgttcagcagaaggcctttctttgggaactgggactctgctgctggggcaaagggtgggcagagtcatgcttgtgctggggacaaaatgaccttgggacacggggcttggctgccacggccggcccgggacagtcggagagtcaggtttttgtgcaccccttaatggggcctcccacaatgtggctactttgactactggggccaagggaccctggtcaccgtctcctcaggtgagtcctcacaacctctctcctcctttaactctgaagggttttgttgacttttgggggaataagggtgctgggggcctgccaagagagccccggagcagccctgggggctgcaggaggcctgaggcaacagcggcacacacagacgaggggcaagggtctccagatgctccttcctcctgagccagcagcacgggttcgtctcggcgccagggccaccctaggcggaggttcgtgtcttctctgagccaggagcacgggttctctcgcaggcaccctgtgcctctggggtccaatgcccaacaaccccggccctccccgggctcagtctgagagggtcccagggacgtgcggggcgccggttctttgtcggggtctggcattgttgtcacaatgtgacaactggttcgactcctggggccaaggaaccctggtcaccgtctcctcaggtgagtcctcaccaccccctctctgagtccacttagggagactcagcttgcagggtct

SEQ ID NO: 45 - protein, 259 aa, Artificial Sequence
  HuC825 anti DOTA scFv
HVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 46 - protein, 39 aa, Artificial Sequence
  P53
KPLDGEYFTLQIRGRERFEMFRELNEALELKDAQAGKEP

SEQ ID NO: 47 - protein, 20 aa, Artificial Sequence
  (G4S)4 linker
GGGGSGGGGSGGGGSGGGGS

SEQ ID NO: 48 - protein, 9 aa, Artificial Sequence
  Linker
TPLGDTTHT

SEQ ID NO: 49 - protein, 7 aa, Artificial Sequence
  Linker
GGSGGAP

SEQ ID NO: 50 - protein, 6 aa, Artificial Sequence
  His tag
HHHHHH

SEQ ID NO: 51 - protein, 97 aa, Artificial Sequence
  IGHV4-4*08
QVQLQESGPGLVKPSETLSLTCTVSGGSISSYYWSWIRQPPGKGLEWIGYIYTSGSTNYNPSLKSRVTISVDTSKNQFSLKLSSVTAADTAVYYCAR

SEQ ID NO: 52 - protein, 16 aa, Artificial Sequence
  IGHJ5*01
NWFDSWGQGTLVTVSS

SEQ ID NO: 53 - protein, 119 aa, Artificial Sequence
  HuOKT3 VH
QVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSS

SEQ ID NO: 54 - protein, 107 aa, Artificial Sequence
  HuOKT3 VL
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 55 - protein, 30 aa, Artificial Sequence
  Linker
GGGGSGGGGSGGGGSGGGGSGGGGSGGGGS

SEQ ID NO: 56 - protein, 300 aa, Artificial Sequence
  CD38 Isoform 1
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 57 - protein, 122 aa, Artificial Sequence
  CD38 Isoform 2
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKK